(19) United States
(12) Patent Application Publication
(10) Pub. No.: US 2011/0207922 A1
(43) Pub. Date: Aug. 25, 2011

(54) MONOSACCHARIDE PREPARATION METHOD

(75) Inventors: Takafumi Kubo, Osaka (JP); Takahiro Inagaki, Osaka (JP); Izuho Okada, Osaka (JP)

(73) Assignee: Nippon Shokubai Co. Ltd, Osaka (JP)

(21) Appl. No.: 13/121,076

(22) PCT Filed: Sep. 28, 2009
(86) PCT No.: PCT/JP2009/066785

§ 371 Date: Mar. 25, 2011

(30) Foreign Application Priority Data
Sep. 29, 2008 (JP) 2008249941
Jan. 13, 2009 (JP) 2009004285
Jun. 23, 2009 (JP) 2009148908

(51) Int. Cl. C07H 1/00; C01G 41/00

(52) U.S. Cl. 536/124; 423/307

(57) ABSTRACT

An object of the present invention is to provide means for preparing a monosaccharide by efficiently hydrolyzing a polysaccharide. In particular, in a method that uses a homogeneous acid catalyst to obtain a monosaccharide from a polysaccharide, a low energy, low cost catalytic separation method is provided, and in addition, a method for obtaining high reaction selectivity is provided. In addition, provided is a homogeneous acid catalyst separation method that separates a homogeneous acid catalyst from a homogeneous acid catalyst-containing solution with high efficiency and realizes a high homogeneous acid catalyst recovery ratio at low energy costs, and that is applicable to a variety of reaction systems.

The present invention is a method for preparing a monosaccharide by hydrolyzing a polysaccharide using a homogeneous acid catalyst,
wherein the method comprises a hydrolysis step of hydrolyzing a polysaccharide using a homogeneous acid catalyst with a molecular weight of 200 or greater to generate a monosaccharide, and a separation step of the homogeneous acid catalyst after hydrolysis, and
the separation step includes at least one step selected from the group consisting of the following (A) to (C):
(A) a step of separating the homogeneous acid catalyst by performing homogeneous acid catalyst membrane separation treatment using a molecular sieve membrane, on a homogeneous acid catalyst-containing solution after the hydrolysis step;
(B) a step of separating the homogeneous acid catalyst by performing organic compound thermal decomposition treatment on a hydrolysis reaction residue separated by solid-liquid separation after the hydrolysis step; and
(C) a step of separating the homogeneous acid catalyst by performing homogeneous acid catalyst elution treatment using an alkaline solution or an organic solvent-containing solution, on the hydrolysis reaction residue separated by solid-liquid separation after the hydrolysis step.

### FIELD OF THE INVENTION

The present invention relates to a monosaccharide preparation method. More specifically, it relates to a monosaccharide preparation method by hydrolysis of polysaccharides, and in particular to monosaccharide preparation method using an acid catalyst of a homogeneous system.

### BACKGROUND ART

In recent years, when crude oil prices are rising steeply, techniques for preparing chemical products such as ethanol and lactic acid from biomass, which is a renewable resource, are drawing attention. As lignocellulosic biomass, which includes polysaccharides such as cellulose and hemicellulose, is present in huge amounts, utilization thereof is promising; however, the utilization is limited to be partial since chemical conversion is difficult. In particular, when chemically converting lignocellulosic biomass, the saccharification reaction of cellulose into glucose is key. As cellulose is high crystalline, accepting little hydrolysis, efficiently saccharifying cellulose has a high level of difficulty. The monosaccharides generated by saccharification are mainly used as raw materials for microbial fermentation, ultimately converted into a chemical product such as ethanol.

As cellulose saccharification methods being examined at the stage of practical use, (1) concentrated sulfuric acid method, (2) dilute sulfuric acid method and (3) enzymatic method may be cited (for instance, refer to Non-patent Document 1 and Non-patent Document 2). The concentrated sulfuric acid method (1) treats cellulose in a high concentration sulfuric acid on the order of 80% at low temperature conditions. Since cellulose dissolves in high concentration sulfuric acid, the present method has the merit that the decomposition reaction proceeds rapidly even at low temperature, and that high monosaccharide yields can be anticipated. However, as there is the need to recover large amounts of sulfuric acid, energy and equipment costs spent for sulfuric acid recovery is an issue. As a prior art sulfuric acid recycling method, one using an ion-exchange resin is known (for instance, refer to Patent Document 1); however, with this method, as sulfuric acid is recovered diluted to on the order of 20%, re-concentration thereof requires considerable energy and equipment. Alternatively, a method using membrane separation with an ion-exchange membrane to recover sulfuric acid is also known (for instance, refer to Patent Document 2); however, also with this method, there is the issue that sulfuric acid is diluted or that the recovery ratio is low. Thus, the concentrated sulfuric acid method has the issue of catalyst recycling, and an economical catalyst recycling method is sought in order to become a highly competitive method.

In addition, the dilute sulfuric acid method (2) treats cellulose in an aqueous solution of sulfuric acid at low concentration, at high temperature and high pressure, and differs fundamentally from the concentrated sulfuric acid method (1) on the points of reaction conditions and decomposition mechanism. Although cellulose dissolves in sulfuric acid of approximately 60% or greater concentration, dissolution does not occur in sulfuric acid of lower concentrations. That is to say, concentrated sulfuric acid method promotes decomposition by dissolving cellulose, in contrast, dilute sulfuric acid method promotes decomposition by bringing it to high temperature and high pressure. With the dilute sulfuric acid method, since the amount of sulfuric acid used is small, no catalyst recycling is carried out; however, there are issues such as low monosaccharide yields, many reaction by-products, waste generated when neutralizing sulfuric acid. Among these, the low yield is the greatest issue. This is due to the low selectivity of the saccharification reaction by the low concentration sulfuric acid, and provision of a catalyst and reaction conditions with high reaction selectivity are required.

The enzymatic method (3) uses an enzyme such as cellulase serving as a catalyst, allowing high yields to be anticipated; however important issues toward practical use are slow reaction speed and high enzyme costs. The above three methods have both advantages and shortcomings, and there is no absolute method currently.

Meanwhile, although in a study stage, a method is being examined, which uses an insoluble heterogeneous solid acid catalyst in the reaction solution to saccharify cellulose (for instance, refer to Patent Document 3). With this method, separation between glucose and catalyst is achieved relatively readily through solid-liquid separation. However, separation between non-decomposed residues such as lignin and the catalyst is difficult, which becomes a problem when decomposing lignocellulose.

In addition, a method that uses a heteropolyacid at a high concentration of on the order of 80% to saccharify cellulose has also been described (for instance, refer to Patent Documents 4 and 5). This method is thought to have a similar mechanism as the concentrated sulfuric acid method, and, while achieving high monosaccharide yields, catalyst recycling is essential. As large amounts of catalyst is used similarly to the concentrated sulfuric acid method, the burden of catalyst recycling is high. In addition, since heteropolyacids are far more expensive compared to sulfuric acid, even a small loss has a large influence on the costs, higher recovery ratio is required. In Patent Document 4, the possibility of using a porous substance such as MFI having 10-membered oxygen ring, the β zeolite or mordenite having 12-membered oxygen ring is described as method for separating a monosaccharide from a catalyst, and in addition, a method for re-precipitating a monosaccharide with an organic solvent is described. In Patent Document 4, an embodiment for recovering heteropolyacid by membrane separation is described, and although phosphotungstic acid is described to have been separated and recovered using a mordenite membrane, there is no mention regarding the recovery ratio for a heteropolyacid such as phosphotungstic acid; adsorption of heteropolyacid onto porous alumina, which is an essential support when using an inorganic membrane, decreases the recovery ratio for heteropolyacid. In addition, in the method for re-precipitating a monosaccharide with an organic solvent, large amounts of solvent is used for the re-precipitation, furthermore, after separation of the catalyst, solvent-removal and dehydration steps are required for concentrating the catalyst, the need for considerable energy and equipment for these steps is a problem. In addition, in the separation method by membrane separation using mordenite membrane, a catalyst dehydration step is also needed after the membrane separation step. In any case, with the methods of Patent Document 4, the burden of catalyst recycling becomes high due to saccharification being carried out with a heteropolyacid at an extremely high concentration, and the energy and equipment costs, furthermore, catalyst costs also, are expected to become considerable.

In addition, a method has been described, which uses an inorganic membrane to membrane-separate a catalyst such as heteropolyacid (for instance, refer to Patent Document 6). Here, a method is given as an example, where vaporizable compound such as ethyl acetate, ethanol, water or acetic acid is separated from heteropolyacid by being vaporized by a method that reduces the pressure on the side of the permeate. However, there are no examples regarding methods for separating a compound that cannot be vaporized, such as a saccharide. In addition, with Patent Document 6, in order to separate a heteropolyacid by a method using an inorganic membrane, which does not permeate the catalyst dissolved in the solution, and reducing the pressure on the permeation side to separate the solvent and the constituents to be eliminated as vapors, gasification of the solvent and the constituents to be eliminated is necessary, at the expense of energy costs. In addition, molecular sieve membranes comprising zeolite or the like are used as the inorganic membranes; however, since the metal oxides constituting the inorganic membranes have the property of adsorbing heteropolyacid, when such inorganic membranes are used to separate heteropolyacid, the heteropolyacid becomes adsorbed, giving rise to losses in the separation and recovery, with an inorganic membrane.

Furthermore, a method has been described, which uses heteropolyacid at low concentration to hydrolyze cellulose (for instance, Non-patent Document 3). Here, silicotungstic acid is used to perform saccharification reaction of cellulose at 60° C. or 100° C. In addition, a method has been described, which similarly uses heteropolyacid at low concentration to hydrolyze cellulose at on the order of 80° C. (for instance, refer to Patent Document 7). These method have a problem in the long reaction time of several tens of hours, and in addition, there is no description regarding heteropolyacid recycling method.

As described above, in the methods for preparing monosaccharides by hydrolyzing polysaccharides such as cellulose, problems exist in the catalyst recycling method, reaction selectivity, and the like, and a proposition is sought, of an efficient and economical process in which these have been solved.

Notice that, in methods for preparing monosaccharides from cellulose, heteropolyacid is used as a catalyst.

Heteropolyacid is an inorganic oxo acid obtained from the condensation of two or more species of oxo acids. Heteropolyacid is anticipated to be used as a homogeneous catalyst in a variety of reactions, and a variety of reactions using this are being examined.

When attempting to use this heteropolyacid industrially, since heteropolyacid per se is expensive, losses between before and after the reaction, even if it is small, have an important influence on the production costs. Thus, after use in the reaction, recycling through separation and recovery is sought. If a heteropolyacid catalyst becomes applied in a variety of reactions and many such reactions are carried out industrially, the importance of heteropolyacid separation/recovery technique increases.

However, owing to the frequent use of heteropolyacid as a homogeneous catalyst, the current situation is that separating and recovering heteropolyacid with a high rate from a reaction solution containing such heteropolyacid is difficult. Thus, such a method that can achieve efficient separation and recovery of heteropolyacid, and furthermore, can be applied to a variety of reaction systems, is desired.

As a prior art catalyst separation techniques, for instance, membrane separation of heteropolyacid using a polyamide reverse-osmosis membrane (for instance, refer to Non-patent Document 4), and recovery of an aggregate containing heteropolyacid using a membrane made of nitrocellulose with a pore size of 3 μm, have been described (for instance, refer to Non-patent Document 5). Or, the possibility of using Nafion to separate and recover, from an aqueous solution of heteropolyacid with a heteropolyacid concentration of 1%, the heteropolyacid (H₃[Mo₁₂O₄₀].3H₂O), has been described (for instance, refer to Non-patent Document 6).

In Non-patent Documents 4 to 6, examples are described, in which a heteropolyacid was separated using an organic polymer membrane that does not require a support. However, in Non-patent Document 4, a reverse-osmosis membrane is used as the membrane, and since a reverse-osmosis membrane in general requires operation at extremely high pressure, the energy cost becomes high, moreover, since the speed of permeation of the permeates is not sufficient, separation efficiency is poor. In Non-patent Document 5, a membrane with a pore size of 3 μm is used, which corresponds to a microfiltration membrane; however, a microfiltration membrane in general is for separating an extremely fine solid, such as a gel, and a liquid, and is unable to separate homogeneously dissolved heteropolyacid. In Non-patent Document 6, Nafion membrane is used as the membrane; however, in addition to the permeation rate for the solvent being remarkably low, separation between heteropolyacid and the solvent is poor.

As described above, while heteropolyacid separation techniques have been described, these are not the techniques in which separation efficiency has been examined closely, merely applying these cannot fully resolve the loss of homogeneous acid catalysts such as heteropolyacid. In addition, these are not separation recovery method that can be considered efficient to the extent of enabling contribution in efficient separation recovery and effective utilization of homogeneous acid catalysts such as heteropolyacid.
Patent Document 1: Japanese Kokai Publication No. 2005-40106 (Specification)
Patent Document 2: WO 2006-085763 (Specification)
Patent Document 3: WO 2008-001696 (Specification)
Patent Document 4: Japanese Kokai Publication No. 2008-271787 (Specification)
Patent Document 5: Japanese Kokai Publication No. 2009-60828 (Specification)
Patent Document 6: Japanese Kokai Publication No. 11-285625 (Specification)
Patent Document 7: Japanese Kokai Publication No. 11-343301 (Specification)
Non-patent Document 1: “Newest technology for Biomass Energy Use” (CMC Publishing Co., Ltd., 2001)
Non-patent Document 2: “Development of New Ethanol Fermentation Technique from Cellulose Biomass/Development of Pretreatment, Saccharification and Ethanol Fermentation Technique” (NEDO Research Report 2005)
Non-patent Document 3: KENICHIRO ARAI and one other, Journal of Applied Polymer Science, Vol. 30, 3051-3057 (1985)
Non-patent Document 4: M. A. FEDOTOV and five others, “Catalysis Letters” (USA), 1990, vol. 6, pp. 417-422
Non-patent Document 5: Chiyo Matsubara and two others, “ANALYST” (UK), 1987, vol. 112, pp. 1257-1260
Non-patent Document 6: S. Roy Chowdhury and three others, “Desalination” (Neth), 2002, Vol. 144, pp. 41-46

### SUMMARY OF THE INVENTION

### Problem to be Solved by the Invention

The present invention was devised in view of the current situation described above, and an object is to provide means for preparing a monosaccharide by efficiently hydrolyzing a polysaccharide. In particular, in a method that uses a homogeneous acid catalyst to obtain a monosaccharide from a polysaccharide, a low energy, low cost catalytic separation method is provided, and in addition, a method for obtaining high reaction selectivity is provided. In addition, an object is to provide a homogeneous acid catalyst separation method that separates a homogeneous acid catalyst from a homogeneous acid catalyst-containing solution with high efficiency and realizes a high homogeneous acid catalyst recovery ratio at low energy costs, and that is applicable to a variety of reaction systems.

### Means for Solving the Problem

As a result of earnest studies, the present inventors discovered that, in a method for preparing a monosaccharide by hydrolyzing a polysaccharide, separating a catalyst with low energy and low cost was possible when a catalyst having a molecular weight of 200 or greater is used, the homogeneous acid catalyst is separated after the hydrolysis reaction, and the separation is carried out by at least one method among (A) method for separating the homogeneous acid catalyst by performing homogeneous acid catalyst membrane separation treatment using a molecular sieve membrane, on a homogeneous acid catalyst-containing solution after the hydrolysis step, (B) method for separating the homogeneous acid catalyst by performing organic compound thermal decomposition treatment on a hydrolysis reaction residue separated by solid-liquid separation after the hydrolysis step, and (C) method for separating the homogeneous acid catalyst by performing homogeneous acid catalyst elution treatment using an alkaline solution or an organic solvent-containing solution, on the hydrolysis reaction residue separated by solid-liquid separation after the hydrolysis step. It was found that this allowed the monosaccharide, which is the product, and the catalyst to be separated and recovered sufficiently, and as a result, also allowed the reaction yield of the monosaccharide to be raised; at the same time, it was also found that, by using heteropolyacid as the homogeneous acid catalyst, setting the mass proportion of homogeneous acid catalyst and water in the hydrolysis reaction to a specific range, or setting the reaction temperature of the hydrolysis reaction to a specific range, allowed the hydrolysis from a polysaccharide to a monosaccharide to proceed more efficiently, which leads to a preparation method allowing a monosaccharide to be generated with higher reaction selectivity.

Furthermore, the present inventors examined, among the methods for separating catalysts, methods using a molecular sieve membrane to separate homogeneous acid catalyst, and focused on organic polymer membranes as molecular sieve membranes. It was found that, owing to the existence of organic polymer membranes with diverse pore diameters, by selecting and using an organic polymer membrane according to the molecular size of the homogeneous acid catalyst contained in the homogeneous acid catalyst-containing solution, or, according to the molecular sizes of the homogeneous acid catalyst and of solutes other than the homogeneous acid catalyst when a solute other than the homogeneous acid catalyst is contained in the homogeneous acid catalyst-containing solution, not only the recovery ratio of the homogeneous acid catalyst could be raised, but also, since a large-capacity porous support is not required, in contrast to when an inorganic membrane is used as the separation membrane, the loss of catalyst recovery ratio attributed to the adsorption of catalyst onto the porous support could be avoided. In addition, it was also found that, by using an organic polymer membrane having a pure water permeation rate of 1 g/min/m² or greater at 25° C. and 0.1 MPa, the solvent permeation rate became sufficient, allowing homogeneous acid catalyst to be separated from the homogeneous acid catalyst-containing solution with high efficiency. Since such membrane separation using an organic polymer membrane can efficiently separate homogeneous acid catalyst regardless of the homogeneous acid catalyst concentration of the homogeneous acid catalyst-containing solution or the molecular weight of the homogeneous acid catalyst, it was found to be particular effective in cases where highly effective separation and recovery of homogeneous acid catalyst could not be realized with prior art homogeneous acid catalyst separation methods, such as in case homogeneous acid catalyst is to be separated from a solution with high homogeneous acid catalyst concentration or in case homogeneous acid catalyst contained in the homogeneous acid catalyst-containing solution is monomeric. Furthermore, it was also found that, in case a solute other than homogeneous acid catalyst is contained in the homogeneous acid catalyst-containing solution and the solute other than homogeneous acid catalyst is an organic compound, since an organic polymer membrane has high affinity to the organic compound, homogeneous acid catalyst could be separated readily by filtering the homogeneous acid catalyst-containing solution while still in liquid form. It was found that, when such an organic polymer membrane is used, in separating the homogeneous acid catalyst and other constituents from the homogeneous acid catalyst-containing solution, the homogeneous acid catalyst could be rejected with a high rejection ratio and the other constituents could be permeated with a high permeation ratio without changing the phase of the constituents in the solution, and an efficient separation at low energy costs was possible. As a result, the inventors have come to a conclusion that the above-mentioned problems can be solved well and thus have accomplished the present invention.

The method for preparing a monosaccharide of the present invention is a preparation method having a common technical thinking on the point that a homogeneous acid catalyst is separated by carrying out a specific treatment on a specific target that is a solution after hydrolysis reaction, which contains a homogeneous catalyst.

That is to say, one of the present invention is a method for preparing a monosaccharide constituted with the following (1) as being essential and another of the present invention is a homogeneous acid catalyst separation method constituted with the following (13) as being essential. The preferred modes of the present invention are constituted by any of the following (2) to (12) and (14) and (15), or a combination thereof. Other preferred modes will be described below.

(1) A method for preparing a monosaccharide by hydrolyzing a polysaccharide using a homogeneous acid catalyst, wherein the method comprises a hydrolysis step of hydrolyzing a polysaccharide using a homogeneous acid catalyst with a molecular weight of 200 or greater to generate a monosaccharide, and a separation step of the homogeneous acid catalyst after hydrolysis, and the separation step includes at least one step selected from the group consisting of the following (A) to (C):

(A) a step of separating the homogeneous acid catalyst by performing homogeneous acid catalyst membrane separation treatment using a molecular sieve membrane, on a homogeneous acid catalyst-containing solution after the hydrolysis step;

(B) a step of separating the homogeneous acid catalyst by performing organic compound thermal decomposition treatment on a hydrolysis reaction residue separated by solid-liquid separation after the hydrolysis step; and

(C) a step of separating the homogeneous acid catalyst by performing homogeneous acid catalyst elution treatment using an alkaline solution or an organic solvent-containing solution, on the hydrolysis reaction residue separated by solid-liquid separation after the hydrolysis step.

(2) The method for preparing a monosaccharide according to (1) above, wherein the hydrolysis step is a step of carrying out hydrolysis with a proportion in mass between the homogeneous acid catalyst and water present in a reaction system in the range of 0.1:99.9 to 50:50, during the hydrolysis reaction.

(3) The method for preparing a monosaccharide according to (1) or (2) above, wherein the homogeneous acid catalyst includes an organic compound having a sulfonic acid group and/or a heteropolyacid.

(4) The method for preparing a monosaccharide according to any of (1) to (3) above, wherein the homogeneous acid catalyst includes a heteropolyacid.

(5) The method for preparing a monosaccharide according to any of (1) to (4) above, wherein the method comprises a recycling step of recovering and recycling the homogeneous acid catalyst separated in the separation step.

(6) The method for preparing a monosaccharide according to (5) above, wherein the recycling step is carried out immediately after the separation step in the monosaccharide preparation method.

(7) The method for preparing a monosaccharide according to any of (1) to (6) above, wherein hydrolysis is carried out at a reaction temperature of 100° C. or higher in the hydrolysis step.

(8) The method for preparing a monosaccharide according to any of (1) to (7) above, wherein the polysaccharide is a polysaccharide obtained through a pretreatment step including at least one among a desalting step, a delignification step and a hemicellulose removal step.

(9) The method for preparing a monosaccharide according to any of (1) to (8) above, wherein the molecular sieve membrane used in the step of separating the homogeneous acid catalyst by performing the membrane separation treatment is a molecular sieve membrane using an organic polymer membrane and a pure water permeation rate of the organic polymer membrane is 1 g/min/m² or greater at 25° C. and 0.1 MPa.

(10) The method for preparing a monosaccharide according to any of (1) to (9) above, wherein the organic polymer membrane is a nano-filtration membrane or an ultrafiltration membrane.

(11) The method for preparing a monosaccharide according to any of (1) to (10) above, wherein the organic polymer membrane is a polymer membrane having a cation-exchange group.

(12) The method for preparing a monosaccharide according to (11) above, wherein the organic polymer membrane is a polymer membrane having a sulfonic acid group.

(13) A method for separating a homogeneous acid catalyst from a homogeneous acid catalyst-containing solution, wherein the method comprises a step of separating the homogeneous catalyst by performing homogeneous catalyst membrane separation treatment using a molecular sieve membrane, and the molecular sieve membrane is a molecular sieve membrane using an organic polymer membrane, and a pure water permeation rate of the organic polymer membrane is 1 g/min/m² or greater at 25° C. and 0.1 MPa.

(14) The method for separating a homogeneous acid catalyst according to (13) above, wherein the organic polymer membrane is a nano-filtration membrane or an ultrafiltration membrane.

(15) The method for separating a homogeneous acid catalyst according to (13) or (14) above, wherein the organic polymer membrane is a polymer membrane having a cation-exchange group.

The present invention will be detailed below.

The method for preparing a monosaccharide (the monosaccharide preparation method) of the present invention comprises a hydrolysis step of hydrolyzing polysaccharides using a homogeneous acid catalyst with a molecular weight of 200 or greater to generate monosaccharides and a homogeneous acid catalyst separation step after the hydrolysis.

The monosaccharide preparation method of the present invention can be used for preparing glucose, which is a monosaccharide species, from a biomass such as lignocellulose. One example of process flow for preparing monosaccharides from biomass is follows: first, pretreatments such as pulverizing and hot water treatment are carried out on the raw material biomass, and saccharification (hydrolysis) is carried out by adding a homogeneous acid catalyst. From the saccharification solution obtained in this way containing monosaccharides and homogeneous acid catalyst, homogeneous acid catalyst is separated, monosaccharides, which are the products, are obtained, and at the same time, recovery of the homogeneous acid catalyst is carried out. As a method for separating the homogeneous acid catalyst, there is the method of carrying out membrane separation treatment using a molecular sieve membrane on the saccharification solution containing the monosaccharides and the homogeneous acid catalyst. In addition, there is the method of treating the saccharification solution containing the monosaccharides and the homogeneous acid catalyst by solid-liquid separation to separate the reaction residue and the reaction solution, and performing organic compound thermal decomposition treatment on the reaction residue, or, the method of performing a homogeneous acid catalyst elution treatment using an alkaline solution or an organic solvent-containing solution on the reaction residue. When carrying out solid-liquid separation, the homogeneous acid catalyst remaining in the reaction solution can be further separated and recovered by carrying out membrane separation treatment using a molecular sieve membrane on the reaction solution separated from reaction residue.

In addition, membrane separation treatment may be carried out using a molecular sieve membrane on the saccharification solution containing the monosaccharides and the homogeneous acid catalyst, and organic compound thermal decomposition treatment may be performed on the obtained solution containing the homogeneous acid catalyst after separating the monosaccharide, or, homogeneous acid catalyst elution treatment using an alkaline solution or an organic solvent-containing solution may be performed on the reaction residue.

One example of process flow in preparing monosaccharides from biomass is shown in FIG. 1.

Hereafter, the homogeneous acid catalyst separation step of the monosaccharide preparation method of the present invention will be described first, then, the hydrolysis step of generating monosaccharides by hydrolyzing polysaccharides, pretreatment of the polysaccharides, which are reaction starting materials, and the monosaccharides, which are the products, and the like, will be described. Thereafter, the homogeneous acid catalyst separation method of the present invention will be described.

The monosaccharide preparation method of the present invention comprises a hydrolysis step of hydrolyzing polysaccharides using a homogeneous acid catalyst to generate monosaccharides, and a homogeneous acid catalyst separation step after hydrolysis, both of which may be carried out once, or may be carried out two or more times. In addition, other steps may be included as long as these steps are included.

While the homogeneous acid catalyst separation step after hydrolysis includes at least one selected from the group consisting of (A) a step of separating the homogeneous acid catalyst by performing homogeneous acid catalyst membrane separation treatment using a molecular sieve membrane, on the homogeneous acid catalyst-containing solution after the hydrolysis step; (B) a step of separating the homogeneous acid catalyst by performing organic compound thermal decomposition treatment on the hydrolysis reaction residue separated by solid-liquid separation after the hydrolysis step; and (C) a step of separating the homogeneous acid catalyst by performing homogeneous acid catalyst elution treatment using an alkaline solution or an organic solvent-containing solution, on the hydrolysis reaction residue separated by solid-liquid separation after the hydrolysis step, it may also include two or more of these. In order to increase homogeneous acid catalyst separation efficiency further, it is preferable that two or more from (A) to (C) are included. More preferable is to include (A) and (B).

Since the steps (B) and (C) above are for separating the homogeneous acid catalyst from a hydrolysis reaction residue which has been separated by solid-liquid separation, solid-liquid separation is an essential step when carrying out the steps (B) and (C), whereas the step (A) being for separating the homogeneous acid catalyst from the homogeneous acid catalyst-containing solution, solid-liquid separation is not essential. Consequently, while the solid-liquid separation step is not an essential step in the monosaccharide preparation method of the present invention, it is preferable to carry out the solid-liquid separation step in order to increase the recovery ratios for the monosaccharides and the homogeneous acid catalyst.

As solid-liquid separation methods, pressure filtration (filter press or the like), aspiration filtration, squeeze separation (screw press or the like), centrifugal separation, precipitation separation (decantation or the like), and the like, can be used, with no particular limitation. Among these, pressure filtration and squeeze separation are preferable from the point of processing speed.

In the solid-liquid separation step described above, further washing with water the reaction residue obtained by carrying out solid-liquid separation by filtration or the like is preferable. This allows the monosaccharides remaining within the reaction residue to be recovered in the water used for washing, allowing the yield of monosaccharides to be increased.

Organic compounds such as non-decomposed polysaccharides, the catalyst, and the like are contained in the reaction residue. With the step (B), the homogeneous acid catalyst is separated by performing a thermal decomposition treatment of the organic compound.

It is preferable that the temperature for the thermal decomposition treatment is 300 to 2,000° C. If lower than 300° C., the organic compound may not be decomposed and eliminated sufficiently. If higher than 2,000° C., the catalyst may be decomposed. More preferable is 350 to 1,000° C., and even more preferable is 400 to 600° C.

In addition, while the duration of the thermal decomposition treatment may be set suitable according to the amount of the reaction residue, 1 to 1,000 minutes is preferable. If shorter than 1 minute, the organic compound may not be eliminated sufficiently. If longer than 1,000 minutes, the efficiency of the separation step decreases. More preferable is 5 to 500 minutes, and even more preferable is 10 to 200 minutes.

The step (C) above is a step of eluting the homogeneous acid catalyst by adding, an alkaline solution or an organic solvent-containing solution to the reaction residue that has been separated by the solid-liquid separation. Either one species from alkaline solution and organic solvent-containing solution may be used, or the alkaline solution and the organic solvent-containing solution may be mixed and used.

As solutions to be used in the step of eluting the homogeneous acid catalyst, while either of an alkaline solution and an organic solvent-containing solution may be used, it is preferable to use an organic solvent-containing solution. If the organic solvent-containing solution is used, separation can be performed without the acid catalyst being neutralized. When the alkaline solution is used, although the acid catalyst becomes neutralized, the catalyst can be separated with a high recovery ratio.

When eluting the homogeneous acid catalyst, it is preferable that the amount of solution used with respect to 100 mass % (solid content) of reaction residue is 10 to 10,000 mass %. If the solution is less than 10 mass %, the catalyst may not be eluted sufficiently. If the solution is more than 10,000 mass %, the catalyst concentration decreases extremely. More preferable is 50 to 1,000 mass %, and even more preferable is 100 to 500 mass %.

For the above alkaline solution, a solution of one or two or more species of alkaline compounds such as sodium hydroxide, potassium hydroxide, calcium hydroxide and magnesium hydroxide can be used. Among these, sodium hydroxide and calcium hydroxide are preferable. More preferable is sodium hydroxide.

As organic solvents used in the above organic solvent-containing solution, one or two or more species from acetone, ethanol, butanol, propanol, methanol, diethyl ether, tetrahydrofuran, methyl ethyl ketone, hexane, and the like, can be used. Among these, acetone, ethanol, butanol and diethyl ether are preferable. More preferable is acetone.

As long as it is an alkaline solution, the above alkaline solution may contain other constituents than the above alkaline compound. As other constituents, for instance, water and organic solvent may be cited. As organic solvents, those mentioned above, and the like, may be cited. The organic solvent-containing solution also, as long as it contains the organic solvent, may contain other constituents. As other constituent, water may be cited.

In the alkaline solution, the content of alkaline compound is preferably 0.01 to 10 mass % and more preferably 0.1 to 5 mass % when the entirety of the alkaline solution is 100 mass %.

In the organic solvent-containing solution, the content of organic solvent is preferably 10 to 100 mass % and more preferably 30 to 80 mass % when the entirety of the organic solvent-containing solution is 100 mass %.

Next, the step (A) above and the homogeneous acid catalyst will be described.

Membrane separation in the present invention is for separating the catalyst and monosaccharides, which are the products, using a separation material having a membrane shape (separation membrane). Separation membranes can be classified according to the separation principles thereof, for instance, classification is possible into those based on the molecular weight differences, those based on differences in ionicity, those based on hydrophilicity-hydrophobicity differences, and the like. The separation membrane used in the present invention is based on the molecular weight differences. Separation membrane based on molecular weight differences is in other words a molecular sieve membrane, the separation membrane used in the present invention is thus a molecular sieve membrane. A molecular sieve membrane is a porous membrane and separates compounds according to the size of the pores thereof.

As parameters representing the properties of a molecular sieve membrane, molecular weight cut-off and pore diameter may be cited. The molecular weight cut-off represents the lowest molecular weight that the separation membrane can reject. In the present invention, the molecular weight of a molecule of which 90% is rejected by the separation membrane defined as the molecular weight cut-off. In addition, in the present invention, molecular weight cut-off of the separation membrane is 500,000 or less from the aspect of separation efficiency. More preferable is 300,000 or less, even more preferable is 100,000 or less and a range of 200 to 100,000 is most preferable. As the pore diameter of the separation membrane, on average, 0.01 to 1,000 nanometers is preferable, 0.05 to 500 nanometers is more preferable, and 0.1 to 100 nanometers is even more preferable.

As species of the molecular sieve membrane, ultrafiltration membrane, dialysis membrane, nano-filtration membrane, reverse-osmosis membrane may be cited, preferably ultrafiltration membrane and nano-filtration membrane, and most preferably nano-filtration membrane.

As materials for the molecular sieve membrane, organic membranes such as carbon membrane, regenerated cellulose, cellulose acetate, nitrocellulose, polyvinylidenefluoride, polytetrafluoroethylene, polysulfone, polyether sulfone, polyacrylonitrile, polyvinyl chloride, aramide, polyimide, aromatic polyamide, hydrophilized polyamide, polyester, polyethylene oxide, polyvinyl alcohol, polyethylene, polyvinylacetate, polyamino acid, and, these with a cation-exchange group introduced; and inorganic membranes such as zeolite, alumina, silica, silicalite, and silicone may be cited. Preferred are those with high stability against acid, heat and pressure, preferably, carbon membrane, regenerated cellulose membrane, cellulose acetate membrane, polysulfone membrane, polyether sulfone membrane, aromatic polyamide membrane, hydrophilized polyamide membrane, zeolite membrane, alumina membrane and silica membrane. Among these, organic membranes with particularly high stability, such as regenerated cellulose membrane, cellulose acetate membrane, polysulfone membrane, polyether sulfone membrane, aromatic polyamide membrane, hydrophilized polyamide membrane, and, these with a cation-exchange group introduced, are preferable.

As shapes of the molecular sieve membrane, tube-shape, bag-shape, hollow fiber-shape, flat membrane-shape, spiral-shape, and the like, may be cited. Preferable is tube-shape, flat membrane-shape, hollow fiber-shape, spiral-shape. More preferable is spiral-shape. As thickness of the membrane, 10 mm or less is preferable, 1 mm or less is more preferable and 0.1 mm or less is even more preferable.

As the molecular sieve membrane, concretely, the following may be cited. Ultrafiltration membranes manufactured by Pall Corporation: Omega series, Alpha series; ultrafiltration membranes manufactured by Asahi Kasei Chemicals Corporation Microza AP series, Microza SP series, Microza AV series, Microza SW series and Microza KCV series; ultrafiltration membranes manufactured by Nitto Denko Corporation: NTU-2120 and RS50; nano-filtration membranes manufactured by Nitto Denko Corporation: NTR-7250, NTR-7259, NTR-7410 and NTR-7450; reverse-osmosis membranes manufactured by Nitto Denko Corporation: NTR-70, NTR-759, ES-40, ES-20, ES-15, ES-10, LES90 and LF-10; ultrafiltration membranes manufactured by Millipore Corporation: Biomax membrane and Ultracell membrane; ultrafiltration membranes manufactured by Daicen Membrane-Systems Ltd.: NADIR UH series, NADIR UP series, NADIR US series, NADIR UC series and NADIR UV series; nano-filtration membranes manufactured by Daicen Membrane-Systems Ltd.: NADIR NP010 and NADIR NP030; reverse-osmosis membranes manufactured by Daicen Membrane-Systems Ltd.: NADIR SW; nano-filtration membranes manufactured by Toray Industries, Inc.: SU series; reverse-osmosis membranes manufactured by Toray Industries, Inc.: SU series, SUL series and SC series; ultrafiltration membranes manufactured by GE Water and Process Technologies: G series membrane, P series membrane and MW series membrane; nano-filtration membranes manufactured by GE Water and Process Technologies: DESAL series; commercial ceramic membranes manufactured by NGK Insulators, Ltd.; and nano-filtration membranes manufactured by Koch Membrane Systems: MPT series and MPS series.

Among the above molecular sieve membranes, preferable are Omega series, Microza AV series, Microza SW series, RS50, NTR-7250, NTR-7259, NTR-7410, NTR-7450, Biomax membrane, NADIR UH series, NADIR UP series, NADIR US series, NADIR UC series, NADIR UV series, NADIR NP010, NADIR NP030, SU series, G series membrane, P series membrane, MW series membrane, DESAL series, MPS series and ceramic membranes manufactured by NGK, more preferable are NTR-7410, NTR-7450, NADIR NP010, NADIR NP30, G series membrane, DESAL series, MPS series and ceramic membranes manufactured by NGK, and even more preferable are NTR-7410, NTR-7450, G series membrane, DESAL series and MPS series.

In the present invention, a homogeneous acid catalyst refers to an acid catalyst that is homogeneous and dissolves in a reaction solution homogeneously. As a homogeneous acid catalyst, higher acidity is preferable from hydrolytic activity view point. As a concrete indication, when the acid catalyst is dissolved in water at a concentration of 5 mass %, an aqueous solution pH is preferably 4 or lower, more preferably 3 or lower, and even more preferably 2 or lower.

The molecular weight of the acid catalyst is 200 or greater. This is due to the molecular weight of the monosaccharide, which is the product, being on the order of 150 to 200. As the molecular weight range, 200 to 500,000 is preferable, 300 to 300,000 is more preferable and 300 to 100,000 is even more preferable. In addition, a difference between the molecular weight of the acid catalyst and the molecular weight cut-off of the molecular sieve membrane of 100 or greater is preferable, 1,000 or greater is more preferable and 3,000 or greater is even more preferable.

The present invention is also a monosaccharide preparation method using a homogeneous acid catalyst having a molecular weight of 200 or greater as the homogeneous acid catalyst. Using such a catalyst allows the catalyst separation to be more efficient and economical.

In the present invention, the size-relationship for the molecular weight of the homogeneous acid catalyst, the molecular weight cut-off of the separation membrane and the monosaccharide molecular weight is: molecular weight of acid catalyst>molecular weight cut-off of separation membrane>molecular weight of monosaccharide. In addition, when a solute other than homogeneous acid catalyst is contained in the homogeneous acid catalyst-containing solution, a preferable size-relationship is molecular weight of homogeneous acid catalyst>molecular weight cut-off of separation membrane>molecular weight of solute other than homogeneous acid catalyst. When membrane separation is performed with such conditions, the catalyst stays on the feed solution side (concentrate side) without permeating the membrane, and the solute other than the homogeneous acid catalyst and the solvent permeate the membrane, moving on the permeate side. The catalyst on the concentrate side is not readily diluted with a solvent such as water, and can be recovered in high concentrations. Furthermore, if concentration of the catalyst is necessary, it can be concentrated as-is by membrane separation, with low energy. With the method of recovering with an ion-exchange resin or the method of recovering with an ion-exchange membrane of the prior art, there is the problem that sulfuric acid is recovered diluted, requiring considerable energy for re-concentration of sulfuric acid, or high recovery ratio is not obtained. With the membrane separation method of the present invention, the merits are that the catalyst can be recovered at high concentration and that the recovery ratio is also high.

In addition, as concentration of the acid catalyst at hydrolysis, 50:50 is the upper limit value in mass proportion of the homogeneous acid catalyst and water present in the reaction system (acid catalyst: water), and it is preferable to carry out the reaction at an acid catalyst concentration that is lower than this. Water referred to here means the total amount of water present in the reaction system, including everything such as moisture contained in the raw materials and added water. While the amount of moisture can be modified by adding or removing water, here, it is defined as the amount of moisture at the beginning of the reaction.

The upper limit value of the mass proportion of the catalyst and water is more preferably 30:70 and even more preferably 20:80. As the lower limit value, 0.1:99.9 is preferable, 0.5:99.5 is more preferable and 1:99 is even more preferable. Note that an acid catalyst concentration of 50:50 in mass proportion becomes 50% when represented in mass %. In the present invention, unless expressly stated otherwise, % represents mass %.

The present invention is also a monosaccharide preparation method in which the above-mentioned hydrolysis is carried out with the mass proportion of the homogeneous acid catalyst and water present in the reaction system in the range of 0.1:99.9 to 50:50.

By carrying out hydrolysis under such conditions, the reaction and catalyst recycling can become more efficient and economical. In addition, setting the proportion of acid catalyst and water in the range of 0.1:99.9 to 50:50 facilitates catalyst separation and recycling.

One of the merits of membrane separation is that the catalyst can be recovered at a relatively high concentration; however, in examination by the present inventors, it was found that due to problems such as liquid viscosity, membrane fouling and corrosion, concentrating the acid catalyst concentration to a high concentration of 50% or greater by membrane separation was substantially extremely difficult. Compared to the concentrated sulfuric acid method or the methods of Patent Document 4 and the like, with the monosaccharide preparation method of the present invention, catalyst concentration at saccharification is low, thus, the burden on catalyst recycling is also low. That is to say, it has the merits that a small amount of catalyst to recycle is sufficient, the time required for membrane separation is short, and worries such as membrane deterioration and fouling decrease. In addition, due to the fact that the catalyst concentration is low, the catalyst solution after membrane separation can be re-used immediately, as-is. With the concentrated sulfuric acid method which re-uses with a concentration of 50% or greater or the method of Patent Document 4 and the like, a dehydration step such as by distillation is necessary; whereas the monosaccharide preparation method of the present invention does not necessarily require such a step, which is also an advantage.

Meanwhile, as described above, methods that carry out saccharification at low catalyst concentration as in the dilute sulfuric acid method and dispose of the catalyst (dilute acid method) have the problem that reaction selectivity is low. This is due to the selectivity of the catalyst being low, rendering the catalyst disposable can also be a cause. That is to say, there is the problem that, in order to render the catalyst disposable, the choices of catalyst species and use conditions are limited. The present inventors discovered that even in dilute acid method conditions, introducing catalyst recycling allows the choices of catalysts to be widened, realizing high reaction selectivity. That is to say, the present invention is a process that introduces a high-performance saccharification catalyst and an efficient catalyst recycling method, at relatively low catalyst concentration. Since an excellent reaction selectivity is realized and the burden of catalyst recycling is low, the method of the present invention is a breakthrough that can realize high economy.

As concrete compounds of the acid catalysts, organic compounds having a sulfonic acid group, organic compounds having a carboxylic acid group, polyacids such as homopolyacids and heteropolyacids may be cited, preferably organic compounds having a sulfonic acid group, and, heteropolyacids, which have high acid strength. That is to say, it is preferable that the homogeneous acid catalyst of the present invention contains an organic compound having a sulfonic acid group, and/or, a heteropolyacid. There are advantages such as the sulfonic acid-containing compounds are available in a variety of molecular weights, and that a heteropolyacid has homogeneous molecular weight. That is to say, it is one preferred embodiment of the present invention that the homogeneous acid catalyst contains an organic compound having a sulfonic acid group, and/or, a heteropolyacid.

The organic compound having a sulfonic acid group refers to an organic compound having at least one sulfonic acid group within the molecule. Concretely, naphthalene sulfonic acid, pyrene sulfonic acid, lignin sulfonic acid and the like may be cited; the compound may have one or a plurality of sulfonic acid groups, and in addition, the compound may have substituent group other than sulfonic acid group. In addition, polymers obtained when sulfonic acid group-containing monomers such as vinyl sulfonic acid, styrene sulfonic acid, sulfomaleic acid and allyloxy-hydroxy-propanesulfonic acid are polymerized or copolymerized with monomers such as acrylic acid and maleic acid may also be cited. Or, polymers obtained by sulfonation of polymers such as polystyrene, polyethylene, polypropylene and polyvinyl alcohol may also be cited. Among these, lignin sulfonic acid and various sulfonic acid group-containing polymers are preferable, and more preferably, various sulfonic acid group-containing polymers. Preferred sulfonic acid group-containing polymers include polymers obtained by polymerizing vinyl sulfonic acid and styrene sulfonic acid, and polymers obtained by copolymerizing vinyl sulfonic acid and styrene sulfonic acid with acrylic acid and/or maleic acid.

The organic compound having a sulfonic acid group may be used alone or may be used by combining two or more species.

As the heteropolyacids, phosphotungstic acids such as Keggin-type phosphotungstic acid (H₃PW₁₂O₄₀) and Dawson-type phosphotungstic acid (H₆P₂W₁₈O₆₂), silicotungstic acids such as Keggin-type silicotungstic acid (H₄SiW₁₂O₄₀), borotungstic acids such as Keggin-type borotungstic acid (H₅BW₁₂O₄₀), phosphomolybdic acid, silicomolybdic acid, phospho vanado tungstic acid, silico vanado tungstic acid, phospho vanado molybdic acid, silico vanado tungstic acid, metal-substituted heteropolyacids, and the like, may be cited. Among these, from the point of view of catalytic activity in various reactions, phosphotungstic acid, silicotungstic acid, borotungstic acid, phosphomolybdic acid and silicomolybdic acid are preferable. Phosphotungstic acid and silicotungstic acid are more preferable, and phosphotungstic acid is even more preferable.

In addition, they can have a salt structure in which a portion of the protons has been substituted by cationic species. In this case, for the cationic species, for instance, sodium, magnesium, ammonium, and the like, may be cited with no particular limitation.

The heteropolyacids and salts thereof may be used alone or may be used by combining two or more species.

The present inventors discovered that, in the hydrolysis reaction of polysaccharides at a low catalyst concentration of 50% or lower, heteropolyacids demonstrate specifically high selectivity compared to other catalysts such as sulfuric acid. In particular, phosphotungstic acid was found to demonstrate high selectivity. In addition, it was found that by combining a saccharification reaction at low catalyst concentration and the three catalyst separation methods described in the present invention, the process becomes realistic even when expensive catalysts such as heteropolyacids are used. That is to say, by using a catalyst solution at 50% or lower, important merits are obtained, of the burden in catalyst separation being alleviated and the cost increase due to a loss in catalyst being decreased as well.

These acid catalysts may be used alone or may be used by combining a plurality. In addition, it may have a salt structure in which a portion of the protons has been substituted by a cation such as sodium, magnesium and ammonium.

In the following, the polysaccharides, which are the reaction starting materials used in the monosaccharide preparation method of the present invention, monosaccharides, which are the products, pretreatment of the polysaccharides, which are the raw materials, the hydrolysis step of generating monosaccharides from polysaccharides, and the like, will be described.

As polysaccharides used in the monosaccharide preparation method of the present invention, lignocellulose, cellulose; and hemicelluloses such as xylan, arabinan, mannan and galactan; chitin, chitosan, agarose, alginic acid, carrageenan, β glucan, and, starch and the like may be cited. Lignocellulose, cellulose, hemicelluloses are preferable and lignocellulose, cellulose are more preferable. Lignocellulose refers to cellulose substance and hemicellulose substance containing lignin, which is a biomass that is present in large quantities in plants.

As origins of the polysaccharides, biomass derived from plants such as needle-leaved trees, broad-leaved trees, herbaceous plants, palms, algae and seaweeds and microorganisms is preferable. Concretely, biomasses such as waste wood or old paper derived from needle-leaved trees and broad-leaved trees, sugar cane (bagasse, leaves), corn (core, leaves), rice straw, wheat straw, switchgrass, oil palm (trunks, leaves, empty fruit bunches, kernel press cake), algae (cell walls, intracellular solid contents) and seaweeds (cell walls, intracellular solid contents) are preferable. More preferable are trunks, leaves, empty fruit bunches and kernel press cake of palms such as oil palm, and cell walls and intracellular solid contents of algae, and even more preferable are empty fruit bunches of palms, and cell walls and intracellular solid contents of algae. Since empty fruit bunches of palms are discarded in large quantities, they are readily available, and algae have the merit of being decomposed readily as they do not contain lignin. Polysaccharides may be pretreated by pulverizing, drying and the like, and used in the reaction.

It is preferable that salts, lignin or hemicellulose present in the raw material polysaccharide, be eliminated through pretreatment step before use. Such step of eliminating salt, lignin or hemicellulose is defined as desalting step, delignification step and hemicellulose removal step, respectively. The present invention is also a monosaccharide preparation method, in which the polysaccharides are those obtained through a pretreatment step including at least one among a desalting step, a delignification step and a hemicellulose removal step. Natural biomass such as lignocellulose contain in general various salts, and when these salts are mixed with the acid catalyst, a salt exchange occurs. Since a salt exchange leads to a change in the catalyst species and a drop in the acid strength, removal of salts as much as possible is preferable.

The present inventors found that, in particular when a heteropolyacid is used as the catalyst for biomass saccharification, the catalyst becomes insoluble due to salt exchange, leading to an extreme drop in activity or causing a loss of catalysis. It is believed that this is due to a substitution with potassium, calcium, ammonium ion or the like. In order to avoid such a precipitation, it is preferable to use polysaccharides that have undergone a desalting step.

In addition, since lignin sometimes adsorb a homogeneous acid catalyst, if lignin is present in the reaction starting materials, it causes a drop in sugar yield or a drop in the recovery ratio of the catalyst. By removing lignin in the delignification step prior to the hydrolysis reaction, the sugar yield during hydrolysis can be increased, and the recovery ratio of the catalyst after hydrolysis can be increased.

In addition, the molecular weight of lignin sometimes drops, causing inhibition of fermentation. By eliminating lignin, inhibition of fermentation can be avoided.

In addition, hemicellulose contained in biomass such as lignocellulose decomposes at a lower temperature than crystalline cellulose. Therefore, when performing the present invention for decomposition of cellulose, if hemicellulose is present in the raw materials polysaccharide, by-products such as furfural are generated by overdegradation. As this will cause a drop in the yield of monosaccharides derived from hemicellulose and inhibition of fermentation due to furfural or the like, it is preferable to remove hemicellulose beforehand.

As the desalting step, method of eliminating by elution with a solvent such as water, method of eliminating by further adding acid or alkali to solvent so as to combine elution and acid decomposition or alkaline decomposition, and the like, may be cited. The elution may be accelerated by heating. Preferred are the method of eliminating by eluting in hot water and the method of eliminating by eluting in hot water added with an acid or an alkali. These methods may be carried out alone, or may be carried out by combining two or more.

As acids used in the desalting step, mineral acids such as sulfuric acid, hydrochloric acid, nitric acid, phosphoric acid, polyacid and carbonic acid; organic acids such as acetic acid and sulfonic acid; and the like, are preferable. As alkali, sodium hydroxide, potassium hydroxide, calcium hydroxide, magnesium hydroxide, ammonia and the like are preferable. Among these, sulfuric acid, carbonic acid, hydrochloric acid, sodium hydroxide, ammonia are more preferable, and sulfuric acid and sodium hydroxide are even more preferable.

In the desalting step, it is preferable to add a solvent to the raw materials polysaccharides and then elute the salts at a temperature of 10 to 200° C. By treating at such temperatures, salts can be eluted sufficiently. More preferable is 20 to 150° C. and even more preferable is 50 to 120° C.

In addition, it is preferable that the duration of the treatment for eluting the salts is 0.01 to 10 hours. More preferable is 0.05 to 3 hours and even more preferable is 0.1 to 1 hour.

In the desalting step, of the salts present in the raw materials prior to the desalting step, removing 50% or more is preferable, removing 80% or more is more preferable, and removing 90% or more is even more preferable. The salt content can be determined by ash measurement, X-ray fluorescence measurement, ion chromatography method, ICP (inductively coupled plasma) emission spectrometry and the like.

As the delignification step, method of eliminating by eluting with an alkaline aqueous solution and the method of eliminating by elution with a solution containing an organic solvent are preferable. An acid or an alkali may be added to the organic solvent. Adding acid or alkali allows lignin decomposition to be promoted. In addition, elution and decomposition may be promoted by heat.

As the acid or the alkali used in the above delignification step, same ones to those used in the above desalting step can be used. As organic solvents used in the delignification step, acetone, ethanol, butanol, methanol, propanol, methyl ethyl ketone, tetrahydrofuran, hexane, toluene and the like can be used. Among these, acetone, ethanol and butanol are preferable, and acetone is more preferable.

In the delignification step, after adding the solution into the raw materials polysaccharides, it is preferable that a treatment is carried out at temperature of 10 to 200° C. Treating at such temperatures allows lignin to be eluted sufficiently. More preferable is 50 to 180° C. and even more preferable is 80 to 150° C. In addition as the duration of the treatment, 0.01 to 10 hours is preferable, more preferably 0.05 to 5 hours and even more preferably 0.1 to 2 hours.

In the delignification step, removing 50% or more of the lignin present in the raw materials prior to the delignification step is preferable, removing 80% or more is more preferable, removing 90% or more is even more preferable. The lignin content can be determined according to methods described in, for instance, Handbook of Analytical Chemistry, 4th Ed. (1991, Maruzen).

As the hemicellulose removal step, although similar methods to those of the desalting step can be carried out, more stringent conditions than those for the desalting step are necessary. That is to say, a treatment temperature of 50 to 250° C. is preferable, 100 to 200° C. is more preferable and 120 to 180° C. is even more preferable. In addition, the duration of the treatment of 0.01 to 10 hours is preferable, more preferably 0.05 to 5 hours and even more preferably 0.1 to 2 hours.

In the hemicellulose removal step, removing 50% or more of the hemicellulose present in the raw materials prior to the hemicellulose removal step is preferable, removing 80% or more is more preferable, removing 90% or more is even more preferable. The hemicellulose content can be determined according to methods described in, for instance, Handbook of Analytical Chemistry, 4th Ed. (1991, Maruzen).

The desalting step, the delignification step and the hemicellulose removal step may be carried out separately or may be carried out simultaneously.

As the above pretreatment step, preferred is one containing a desalting step or one containing a hemicellulose removal step, more preferred is one containing a desalting step and a hemicellulose removal step or one containing a hemicellulose removal step and a delignification step, and even more preferred is one containing a desalting step and a hemicellulose removal step.

As monosaccharides in the monosaccharide preparation method of the present invention, those obtained by hydrolyzing the above polysaccharides, concretely, glucose, xylose, arabinose, mannose, galactose, uronic acid, glucosamine, and the like, may be cited. Preferred are glucose and xylose.

As applications of the monosaccharides, utilization as fermentation raw materials, chemical reaction starting materials, fertilizer and feed may be cited, and preferably fermentation raw materials. As fermentation raw materials application, monosaccharides can be used in conversion into alcohols such as ethanol, butanol and 1,3-propanediol; organic acids such as acetic acid, lactic acid, itaconic acid, malic acid, citric acid, acrylic acid and 3-hydroxypropionic acid; various amino acids such as aspartic acid, glutamic acid and lysine, and the like. Among these, preferred is utilization in the preparation of ethanol and butanol, and, acrylic acid and 3-hydroxypropionic acid.

As methods for hydrolyzing polysaccharides in the hydrolysis step of the monosaccharide preparation method of the present invention, one bringing into contact the above acid catalyst and the polysaccharides in the presence of water is adequate, preferably one that mixes an aqueous solution of acid catalyst with the polysaccharides and reacts them. For the reactor type, batch reactor, continuous reactor, semi-continuous reactor, and the like, may be cited, preferably continuous reactor. An organic solvent may be mixed during the reaction. As organic solvents, ethanol, butanol, acetone, and the like, may be cited.

While the concentration of the acid catalyst during the above hydrolysis is as described above, when the mass proportion is expressed in terms of mass % with respect to the entirety (acid catalyst+water) instead, it is expressed as follows: the preferred upper limit value of the acid catalyst concentration is 50%, 30% is more preferable and 20% is even more preferable. The preferred lower limit value of the acid catalyst concentration is 0.1%, 0.5% is more preferable and 1% is even more preferable. Conversely, the preferred upper limit value of water concentration is 99.9%, 99.5% is more preferable and 99% is even more preferable. The preferred lower limit value of the water concentration is 50%, 70% is more preferable and 80% is even more preferable.

In addition, as concentration of the above raw materials polysaccharides in mass % of raw materials polysaccharides with respect to the total amount of reactants, 70% is preferable as the upper limit value, 60% is more preferable and 50% is even more preferable. As the lower limit value, 1% is preferable, 5% is more preferable and 10% is even more preferable. Here total amount of reactants is the mass including everything, such as raw materials polysaccharides, acid catalyst, water, and other solvents. The mass of raw materials polysaccharides means dry mass.

As the reaction temperature of the hydrolysis, a lower limit value of 20° C. is preferable, 100° C. is more preferable and 150° C. is even more preferable. As the upper limit value of the reaction temperature, 300° C. is preferable, 270° C. is more preferable and 250° C. is even more preferable. The present invention is also a monosaccharide preparation method in which the hydrolysis is carried out at a reaction temperature of 100° C. or higher. The present inventors discovered that with a reaction temperature of 100° C. or higher, sufficiently high reaction rate could be obtained even with a low concentration of catalyst, which gives rise to a realistic process. In addition, it was discovered that by elevating the reaction temperature, not only the high reaction rate but the selectivity of the monosaccharides also increased. This was particularly remarkable in the biomass hydrolysis reaction using a heteropolyacid.

In addition, the present inventors discovered that merits were obtained also in the membrane separation step by raising the reaction temperature. That is to say, if there action temperature is raised, generation of reactive by-products such as furfural and formic acid can be suppressed. These reactive compounds provoke problems such as reacting with the separation membrane and accelerating membrane deterioration, or polymerizing to form a polymer compound and provoke fouling of the membrane, and becoming impossible to separate from the catalyst. Consequently, raising the reaction temperature also leads to increasing the life span of the membrane and stable conduction of membrane separation.

As the reaction pressure of the hydrolysis, a lower limit value of 0.01 MPa is preferable, 0.03 MPa is more preferable and 0.05 MPa is even more preferable. As the upper limit value of the reaction pressure, 100 MPa is preferable, 70 MPa is more preferable and 50 Mpa is even more preferable. As the reaction solution pH, pH 4 or lower is preferable, pH 3 or lower is more preferable and pH 2 or lower is even more preferable.

As the reaction time of the hydrolysis, 0.1 to 1,000 minutes is preferable. If the reaction time is shorter than 0.1 minutes, hydrolysis of monosaccharides cannot proceed thoroughly, and the yield of monosaccharides may not be sufficient. In addition, if the reaction time is longer than 1,000 minutes, overdegradation of monosaccharides occurs, and the selectivity of monosaccharides may decreases. More preferable is 0.2 to 200 minutes and even more preferable is 0.3 to 60 minutes.

The hydrolysis reaction may be carried out in multiple stages. In particular, hydrolysis of lignocellulose is preferably carried out in multiple stages. This is due to a difference in the ranges of decomposition temperature between hemicellulose and cellulose contained in lignocellulose. That is to say, it is preferable that, in a first stage, hemicellulose, which can be decomposed in relatively mild conditions, is decomposed, and in a second stage, changing to more stringent conditions to carry out decomposition of cellulose. The acid catalyst used in the first stage and the second stage may be the same one or may be different ones.

While the membrane separation may be carried out after the hydrolysis step is finished, or simultaneously to the reaction, a method in which the membrane separation is carried out after the reaction is preferable. As method of membrane separation by molecular sieve membrane, method of pressurizing the feed solution side (concentrate side), method of reducing pressure on the permeate side, method of diffusion by osmotic pressure, method by centrifugal separation, method using electric potential difference, and the like, may be cited. And the method of pressurizing the feed solution side, and the method of diffusion by osmotic pressure is preferable, and the method of pressurizing the feed solution side is more preferable. In the case of the method of pressurizing the feed solution side, a pressure at the time of membrane separation performance (gauge pressure) is preferably 0.01 MPa to 10 MPa, more preferably 0.03 MPa to 5 MPa and most preferably 0.05 MPa to 4 MPa.

Note that, even a case where membrane separation is carried out simultaneously to the reaction, as long as hydrolysis reaction is carried out for at least a portion of the polysaccharides and monosaccharides are generated in the solution, corresponds to carrying out membrane separation against homogeneous acid catalyst-containing solution after the hydrolysis step in the present invention.

In the present invention, as the type of filtration at the membrane separation performance, either of dead-end type and cross-flow type can be applied; however, the cross-flow type is preferred in the homogeneous acid catalyst separation method of the present invention as separation of the homogeneous acid catalyst can be carried out with high efficiency even if the homogeneous acid catalyst-containing solution is at a high concentration.

Membrane separation in cross-flow type can be carried out by a method whereby, for instance, the solution subjected to separation is pressurized while being sent with a feeding pump to a spiral-shaped separation membrane module to acquire the permeate.

During performance of membrane separation, a temperature of 0° C. to 100° C. is preferable, more preferably 0° C. to 80° C. and most preferably 5° C. to 50° C. While membrane separation may use any method from batch type, continuous type and semi-continuous type, batch type and continuous type are preferable. In order to increase the yield of monosaccharide, membrane separation can be carried out while adding water to the concentrate.

The separated monosaccharide can be used in a fermentation process, via a neutralization step as necessary. In the present invention, as the monosaccharide and the acid catalyst have been separated by membrane separation, the necessity for neutralizing the carbohydrate solution is low, which is also an advantage. That is to say, it is one preferred embodiment of the present invention that the monosaccharide preparation method contains a recycling step of recovering and recycling the homogeneous acid catalyst separated in the separation step. Here, recycling means repeatedly utilizing the catalyst recovered by membrane separation in a hydrolysis reaction.

The acid catalyst concentration of the catalyst solution recovered by the membrane separation is preferably 0.8 times or more the acid catalyst concentration used in the hydrolysis reaction, more preferably 1.0 time or more and even more preferably 1.5 times or more. By bringing the recovered acid catalyst concentration to higher than the acid catalyst concentration during hydrolysis (that is to say by recovering with a concentration of 1.0 time or more), the separated catalyst solution can be recycled immediately. From the foregoing, it is also one preferred embodiment of the present invention that the recycling step is carried out immediately after the membrane separation step. Carrying out the recycling step immediately means carrying out the hydrolysis step again without performing a dehydration step for concentration. Prior to hydrolysis, water may be added to adjust the concentration.

In addition, as the acid catalyst concentration of the recovered catalyst solution, the upper limit value is 50%. Although it depends on the balance with the acid catalyst concentration during hydrolysis, it is more preferably 30%, even more preferably 20% and most preferably 10%. A recovery ratio of the catalyst recovered by membrane separation is preferably 50% or greater, more preferably 70% or greater, even more preferably 90% or greater and most preferably 99% or greater.

Although it is preferable that the recovered catalyst is re-used as-is, if it was subjected to cation-exchange and the proton concentration has dropped, it is preferable to implement a catalyst regeneration step. Here, a regeneration step is to revert the exchanged cation back to protonated form. As methods of regeneration, methods that use cation exchanger are preferable. Concretely, a method whereby a cation exchanger in protonated form and the recovered acid catalyst solution are brought into contact using a column is preferable. As cation exchangers, organic compounds such as cation-exchange resin and inorganic compounds such as zeolite can be used. Preferred are methods that use a cation-exchange resin. Cation exchanger with reduced protons due to cation-exchange can be regenerated by flowing strong acids such as sulfuric acid and re-used.

In this way, in the present invention, an acid catalyst recovered with a molecular sieve membrane can be recycled without undergoing a dehydration step, which is also a major advantage. With the catalyst recovery method using an ion exchange resin or ion-exchange membrane in the concentrated sulfuric acid method, or the methods described in Patent Document 4, the acid catalyst is recovered at a low concentration, furthermore it is necessary to return it to an extremely high concentration. Therefore, there is the problem of requiring considerable energy for re-concentration or low catalyst recovery ratio. In contrast, the method described in the present invention is a lower energy, low cost process.

The monosaccharide preparation method of the present invention is not limited to the above embodiments, and a variety of modifications are possible within the scope indicated in the claims. That is to say, embodiments obtained by combining technical means modified suitably within the range indicated in the Claims are also included within the technical scope of the present invention.

In the following, the method for separating a homogeneous acid catalyst (homogeneous acid catalyst separation method) of the present invention will be described.

The homogeneous acid catalyst separation method of the present invention is a method for separating a homogeneous acid catalyst from a homogeneous acid catalyst-containing solution, wherein the separation method comprises the step of separating a homogeneous acid catalyst by performing a homogeneous catalyst membrane separation treatment using a molecular sieve membrane, and the molecular sieve membrane is a molecular sieve membrane using an organic polymer membrane and the pure water permeation rate of the organic polymer membrane being 1 g/min/m² or greater at 25° C. and 0.1 MPa.

The homogeneous acid catalyst separation method of the present invention comprises the step of separating a homogeneous acid catalyst from a homogeneous acid catalyst-containing solution with a molecular sieve using an organic polymer membrane. As described above, a molecular sieve separates compounds based on the difference in molecular weights, and as long as the homogeneous acid catalyst is separated according to such a principle, the organic polymer membrane used in the homogeneous acid catalyst separation method of the present invention may be one species, or may be two species or more. In addition, the homogeneous acid catalyst separation method may be used in combination with another separation method as long as the homogeneous acid catalyst is separated using at least one organic polymer membrane, and may include another separation step as long as it includes the step of separating using an organic polymer membrane.

Note that, although the homogeneous acid catalyst separation method of the present invention separates a homogeneous acid catalyst from a homogeneous acid catalyst-containing solution using an organic polymer membrane, a separation method corresponds to the separation method of the present invention as long as at least a portion of the homogeneous acid catalyst is separated from any constituent other than the homogeneous acid catalyst contained in the homogeneous acid catalyst-containing solution using an organic polymer membrane. Above all, it is preferable that at least a portion of the homogeneous acid catalyst is separated from the entirety of the constituents other than the homogeneous acid catalyst contained in the homogeneous acid catalyst-containing solution.

The organic polymer membrane used in the homogeneous acid catalyst separation method of the present invention has a pure water permeation rate at 25° C. and 0.1 MPa of 1 g/min/m² or greater. Accordingly, a membrane such as the Nafion membrane described in Non-patent Document 6, which does not permeate pure water under the conditions of 25° C. and 0.1 MPa, does not correspond to the organic polymer membrane in the present invention. A pure water permeation rate of the organic polymer membrane of 1 g/min/m² or greater at 25° C. and 0.1 Mpa gives a membrane with a sufficient solvent permeation rate, allowing the homogeneous acid catalyst to be separated from the homogeneous acid catalyst-containing solution with high efficiency.

It is preferable that the pure water permeation rate is 5 to 1,000 g/min/m². More preferable is 10 to 800 g/min/m². Even more preferable is 20 to 800 g/min/m² and particularly preferable is 30 to 800 g/min/m².

Note that the pure water permeation rate can be determined, for instance, by measuring the flow rate of the permeate obtained when pressurizing to 0.1 MPa in a state where pure water has been flown into the module of each separation membrane.

As homogeneous acid catalysts in the present invention, the organic compounds having a sulfonic acid group, the organic compounds having a carboxylic acid group and the polyacids such as homopolyacids and heteropolyacids described above may be cited, and the preferred conditions when separating the homogeneous acid catalyst for any of these are the same as described above.

The homogeneous acid catalyst separation method of the present invention can be applied preferably when the homogeneous acid catalyst contains a heteropolyacid. As described above, if an inorganic membrane is used when separating a heteropolyacid, since the metal oxide constituting the inorganic membrane has the property of adsorbing the heteropolyacid, separation recovery loss occurs due to adsorption of the heteropolyacid to the inorganic membrane. Furthermore, a porous support is essential when using an inorganic membrane, and since the heteropolyacid also adsorb onto the porous support, this also becomes a cause for separation recovery loss of the heteropolyacid. With the separation method of the present invention, which uses an organic polymer membrane for separation, there is no such loss of heteropolyacid due to adsorption, and recovery is possible with a higher recovery ratio.

From the foregoing, it is one preferred embodiment of the present invention that the homogeneous acid catalyst contains heteropolyacid.

It is preferable that the molecular weight cut-off of the organic polymer membrane and the type of use of the organic polymer membrane are the same as for the molecular sieve membrane used for membrane separation in the monosaccharide preparation method of the present invention described above.

As species of the organic polymer membranes, while those generally called ultrafiltration membranes, dialysis membranes, nano-filtration membranes and reverse-osmosis membranes may be cited, it is preferable that the organic polymer membrane used in the heteropolyacid separation method of the present invention is a nano-filtration membrane or an ultrafiltration membrane. If the organic polymer membrane is a nano-filtration membrane or an ultrafiltration membrane, for instance, when a solute other than heteropolyacid, such as a small molecule organic compound, is contained in the heteropolyacid-containing solution, it is possible to separate the heteropolyacid and the solute other than heteropolyacid. More preferable is a nano-filtration membrane.

As materials of the organic polymer membrane, the same materials as for the molecular sieve membrane used in the membrane separation in the monosaccharide preparation method of the present invention described above may be cited, among which the preferable ones are also the same.

It is preferable that the organic polymer membrane is a polymer membrane having a cation-exchange group. If the homogeneous acid catalyst contains a heteropolyacid, when separation of heteropolyacid from the heteropolyacid-containing solution is carried out using an organic polymer membrane containing a cation-exchange group, the heteropolyacid and the cation-exchange group of the polymer membrane repel each other due to electrical interaction. For this reason, the heteropolyacid cannot approach the polymer membrane readily and passage through the polymer membrane becomes more difficult. This further rejection of the heteropolyacid through the membrane, enabling separation and recovery of heteropolyacid with even less losses. Above all, it is more preferable that the organic polymer membrane is a polymer membrane having a sulfonic acid group.

Concrete examples of organic polymer membranes used in the homogeneous acid catalyst separation method of the present invention and preferable ones among them are the same ones as the organic polymer membranes among the molecular sieve membranes used for the membrane separation in the monosaccharide preparation method of the present invention described above.

In the homogeneous acid catalyst separation method of the present invention, the concentration of homogeneous acid catalyst-containing solution is not limited in particular.

In general, when carrying out membrane separation of a solution, a solution at a low concentration is used, and separation of a solute cannot be carried out sufficiently with a solution at a high concentration. However, in the homogeneous acid catalyst separation method of the present invention, even if the concentration of the homogeneous acid catalyst-containing solution is high, the homogeneous acid catalyst can be separated. Thus, the effects of the present invention are exerted more remarkably when the concentration of the homogeneous acid catalyst-containing solution is high.

It is also one preferred embodiment of the present invention that the concentration of the homogeneous acid catalyst in the homogeneous acid catalyst-containing solution is 1 mass % or greater. As a preferred embodiment of the present invention, more preferable is 2 mass % or greater and even more preferable is 4 mass % or greater.

Note that, in the present invention, the mass of the homogeneous acid catalyst divided by the total mass between the mass of the homogeneous acid catalyst and the mass of the solvent is represented as the concentration of the homogeneous acid catalyst.

As the solvents, while selection is possible according to the application or the like of the homogeneous acid catalyst-containing solution with no particular limitation, for instance, water, various alcohols, various ethers, various esters, and the like, may be cited.

In the homogeneous acid catalyst separation method of the present invention, the size relationship between the molecular weight of the homogeneous acid catalyst and the molecular weight cut-off of the organic polymer membrane is the same as the size relationship between the molecular weight cut-off of the molecular sieve membrane used for membrane separation and the molecular weight of the homogeneous acid catalyst in the monosaccharide preparation method of the present invention described above.

A difference between the molecular weight of the homogeneous acid catalyst and the molecular weight cut-off of the organic polymer membrane is preferably 100 or greater, more preferably 300 or greater and even more preferably 500 or greater.

In addition, as the molecular weight of the homogeneous acid catalyst, 1,000 or greater but 10,000 or less is preferable. While highly efficient separation and recovery of a homogeneous acid catalyst from a homogeneous acid catalyst-containing solution in which the molecular weight of the homogeneous acid catalyst is in such a range has been difficult so far, in the present invention, a homogeneous acid catalyst in such a range can also be separated efficiently. Thus, the effects of the present invention are exerted more remarkably when molecular weight of the homogeneous acid catalyst is in the range mentioned above. More preferably 1,000 or greater but 7,500 or less and even more preferably 1,000 or greater but 5,000 or less.

As described above, it is one preferred embodiment of the present invention that the homogeneous acid catalyst contains a heteropolyacid, and as concrete examples of heteropolyacid, the same as those described above are preferable.

In addition, it is preferable that the method of membrane separation and the pressure during performance of membrane separation in the homogeneous acid catalyst separation method of the present invention are the same as for the membrane separation in the monosaccharide preparation method of the present invention described above.

In addition, it is preferable that separation type, temperature during performance of membrane separation and the type of membrane separation (batch type, continuous type, semi-continuous type and the like) in the homogeneous acid catalyst separation method of the present invention are the same as those for the membrane separation in the monosaccharide preparation method of the present invention described above.

The membrane permeation rate of a permeate in the homogeneous acid catalyst separation method of the present invention can be set according to the concentrations of the homogeneous acid catalyst and other solutes, as well as the pressure (gauge pressure) during performance of membrane separation.

Although the membrane permeation rate of a permeate is not limited in particular except that the upper limit value is limited by the maximum pressure of the separation membrane and the separation membrane module, from the point of view of the homogeneous acid catalyst separation efficiency and rejection ratio described below, 50 g/min/m² or greater is preferable, more preferable is 100 g/min/m² or greater and most preferable is 200 g/min/m² or greater.

Note that the membrane permeation rate of the permeate can be determined, for instance, by measuring the flow rate of the permeate during membrane separation.

In the homogeneous acid catalyst separation method of the present invention, as rejection ratio for the homogeneous acid catalyst, it is preferable that the homogeneous acid catalyst rejection ratio when a homogeneous acid catalyst-containing solution in which the homogeneous acid catalyst concentration exceeds 1 mass % is subjected to membrane separation and the permeate amount has reached 10% of the liquid amount of the solution subjected to membrane separation (initial homogeneous acid catalyst rejection ratio) is 70% or greater. When the initial homogeneous acid catalyst rejection ratio is in such a range, permeation of the homogeneous acid catalyst is prevented sufficiently and it can be deemed that the homogeneous acid catalyst could be separated sufficiently. More preferable is 80% or greater and even more preferable is 85% or greater.

In addition, since the homogeneous acid catalyst separation method of the present invention can separate homogeneous acid catalyst even if the concentration of the homogeneous acid catalyst-containing solution is high, separation of the homogeneous acid catalyst can be carried out without the rejection ratio for the homogeneous acid catalyst dropping even if the separation process proceeds and the solution subjected to membrane separation is becoming concentrated. That is to say, it is also one preferred embodiment of the present invention that the homogeneous acid catalyst rejection ratio, when a homogeneous acid catalyst-containing solution in which the homogeneous acid catalyst concentration exceeds 1 mass % is subjected to membrane separation in the homogeneous acid catalyst separation method of the present invention and the permeate amount has reached 50% of the liquid amount of the solution subjected to membrane separation, is 70% or greater. More preferably as the preferred embodiment, the homogeneous acid catalyst rejection ratio, when the permeate amount has reached 50% of the liquid amount of the solution subjected to membrane separation, is 80% or greater and even more preferably 85% or greater.

Note that the rejection ratio for the homogeneous acid catalyst can be calculated by the following Calculation Formula (1):

R=1−Cp/Cb (1)

In the above Formula (1), R represents the rejection ratio for the homogeneous acid catalyst, Cp represents the homogeneous acid catalyst concentration on the permeate side and Cb represents the homogeneous acid catalyst concentration on the feed solution side, respectively.

In addition, the homogeneous acid catalyst-containing solution subjected to membrane separation in the homogeneous acid catalyst separation method of the present invention may contain a solute other than the homogeneous acid catalyst, and above all, a mode in which the homogeneous acid catalyst-containing solution contains an organic compound with a molecular weight of 1,000 or less is also one preferred embodiment of the present invention. And furthermore, a mode in which the organic compound contains a saccharide is also one preferred embodiment of the present invention.

There is no particular limitation on the concentration of the organic compound with a molecular weight of 1,000 or less contained in the homogeneous acid catalyst-containing solution.

In addition, it is preferable that, when the homogeneous acid catalyst-containing solution containing the organic compound with a molecular weight of 1,000 or less has been separated by the homogeneous acid catalyst separation method of the present invention, the membrane permeation ratio of the organic compound is 70% or greater. If the permeation ratio of the organic compound is in such a range, it can be considered that the organic compound has permeated the organic polymer membrane sufficiently, and from the fact that the organic compound permeates the membrane and permeation of the membrane is prevented for the homogeneous acid catalyst as described above, it can be deemed that the homogeneous acid catalyst and the organic compound and solvent could be separated efficiently enough. More preferable is 80% or greater and even more preferable is 90% or greater.

Note that the membrane permeation ratio of the organic compound can be calculated from the organic compound concentration of the solution subjected to the membrane separation and the organic compound concentration in the permeate.

A high homogeneous acid catalyst recovery ratio can be realized by recovering the homogeneous acid catalyst efficiently separated by the homogeneous acid catalyst separation method of the present invention. A homogeneous acid catalyst recovery method containing such a step of recovering a homogeneous acid catalyst using the homogeneous acid catalyst separation method of the present invention is also one of the present inventions.

As the homogeneous acid catalyst recovery ratio, 70% or greater when the homogeneous acid catalyst-containing solution in which the homogeneous acid catalyst concentration exceeds 1 mass % has been subjected to membrane separation is preferable. More preferable is 80% or greater and even more preferable is 90% or greater.

Note that the homogeneous acid catalyst recovery ratio can be determined as the proportion of the homogeneous acid catalyst amount remaining on the concentrate side after separation with respect to the homogeneous acid catalyst amount contained in the homogeneous acid catalyst-containing solution prior to separation.

The method for separating a homogeneous acid catalyst (the homogeneous acid catalyst separation method) of the present invention is a separation method by way of a molecular sieve using an organic polymer membrane, in which the pure water permeation rate of the organic polymer membrane is 1 g/min/m² or greater at 25° C. and 0.1 MPa. This separation method, owing to the fact that it separates by way of a molecular sieve using an organic polymer membrane, is a separation method for homogeneous acid catalyst that is industrially applicable, without requiring a special operation, to reaction systems that use a variety of homogeneous acid catalysts, in addition to methods of hydrolyzing polysaccharides using a homogeneous acid catalyst to prepare monosaccharides.

As the reaction systems, for instance, oxidation reactions such as epoxidation reaction, alkane oxidation reaction, aromatic branched chain alkyl group oxidation reaction, aromatic hydroxyl group oxidation reaction and alcohol oxidation reaction; acid catalyzed reactions such as isomerization reaction and hydrolysis reaction of olefin, alcohol dehydration reaction, etherification reaction, esterification reaction, Friedel-Crafts reaction, polymerization reaction and hydrolysis reaction including biomass saccharification reaction may be cited. Among these, as one particularly preferred mode that applies the homogeneous acid catalyst separation method of the present invention, in a biomass saccharification method using a homogeneous acid catalyst, application when separating the homogeneous acid catalyst from the homogeneous acid catalyst-containing solution after the saccharification reaction may be cited. Biomass saccharification method is one of the oil substitution energy techniques drawing attention in recent years, and applying the present invention to such a technique has particularly important technical significance as biomass product purification technique and cost reduction technique.

In the preparation of monosaccharides from biomass, saccharides, which are the reaction products obtained by the biomass saccharification reaction, are contained in the homogeneous acid catalyst-containing solution after the reaction, and the homogeneous acid catalyst separation method of the present invention can be used suitably to the step of separating homogeneous acid catalyst and saccharides from such a homogeneous acid catalyst-containing solution. That is to say, it is one preferred embodiment of the homogeneous acid catalyst separation method of the present invention to use the homogeneous acid catalyst separation method of the present invention in the monosaccharide preparation method of the present invention when carrying out the homogeneous acid catalyst separation step by way of the step (A). When using the homogeneous acid catalyst separation method of the present invention in the monosaccharide preparation method of the present invention, it is preferable to use the preferred mode in the homogeneous acid catalyst separation method of the present invention described above.

As the saccharides, for instance, glucose, xylose, arabinose, mannose, galactose, uronic acid, glucosamine, and the like, may be cited.

### Effect of the Invention

The monosaccharide preparation method of the present invention comprises the constitution described above and allows monosaccharides to be prepared efficiently and economically from an inexpensive biomass such as lignocellulose, thus, it is a preparation method that can be used suitably as raw materials to prepare chemical products such as ethanol and lactic acid.

In addition, the homogeneous acid catalyst separation method of the present invention comprises the constitution described above and allowing a homogeneous acid catalyst to be separated from a homogeneous acid catalyst-containing solution with high efficiency and a high homogeneous acid catalyst recovery ratio to be obtained, at low energy cost.

### BRIEF DESCRIPTION OF THE DRAWINGS

FIG. 1 shows one example of process flow for preparing monosaccharides from biomass using a homogeneous acid catalyst, and for recovering the catalyst.

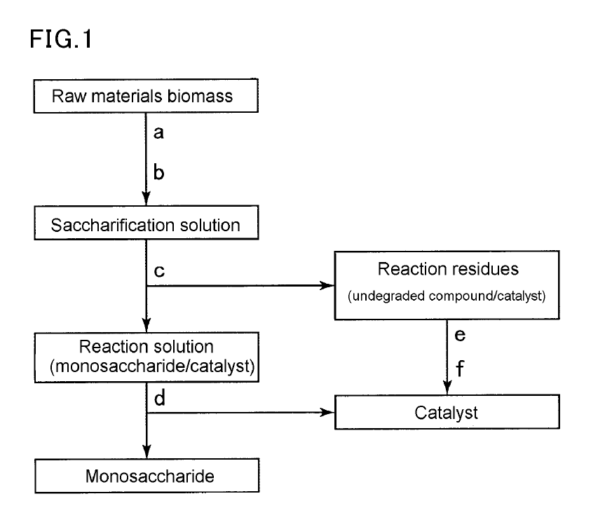

### EXPLANATION OF NUMERAL(S) AND SYMBOL(S)

a: Pretreatment (pulverizing, hot water treatment and the like)
b: Saccharification (hydrolysis of polysaccharides using a homogeneous acid catalyst)
c: Solid-liquid separation
d: Membrane separation treatment (molecular sieve membrane)
e: Thermal decomposition treatment
f: Elution treatment

### BEST MODE FOR CARRYING OUT THE INVENTION

Hereafter, examples will be given to described the present invention in further details; however, the present invention is not limited to these examples only. Note that, unless expressly stated otherwise, “parts” means “parts by weight” and “%” means “mass %”.

The analytical methods and calculation methods used in the examples are shown below.

(Quantification of Monosaccharides)

The quantification of monosaccharides was carried out by liquid chromatography (HPLC). For the column, TSK-GEL Amide80 manufactured by Tosoh Corp. was used, and detection was carried out with a refractometer (RI). The yield of the monosaccharides was calculated according to the formula below:

Monosaccharide yield (mass %)=total mass of generated monosaccharides/mass of raw materials polysaccharides×100

Here, the mass of raw materials polysaccharides is, in the case of cellulose, the dry mass of the raw materials cellulose, and in the case of palm empty fruit bunches (fruit bunches after removal of palm nut; hereafter referred to as palm EFB), the dry mass of raw materials palm EFB.

(Quantification of By-Products)

The quantification of by-products was carried out by HPLC.

For the column, TSK-GEL ODS-100V manufactured by Tosoh Corp. was used, and detection was carried out by using an ultraviolet spectrophotometer (UV) and RI. The monosaccharide selectivity of the saccharification reaction was calculated according to the formula below:

Selectivity (mass %)=total mass of generated monosaccharide/total mass of products (monosaccharides and by-products)×100

By-products are furfural, hydroxymethylfurfural, formic acid, levulinic acid, and acetic acid which are generated by overdegradation of monosaccharides.

(Quantification of Acid)

The concentration of sulfonic acid compound in solution was calculated from the sulfur amount determined by inductively coupled plasma analysis (hereafter, ICP analysis) using ICPE-9000 manufactured by Shimadzu Corp. In addition, the in-solution phosphotungstic acid concentration was calculated from the tungsten amount determined by ICP.

(Catalyst Quantification in Solid)

The amount of phosphotungstic acid present in solid was determined from the tungsten amount determined by X-ray fluorescence measurement (proportion occupied in ash) and the ash amount determined by ash measurement.

(Catalyst Recovery Ratio)

The catalyst recovery ratio was calculated according to the formula below:

Catalyst recovery ratio (mass %)=mass of recovered catalyst/mass of catalyst present prior to recovery×100

### Example 1

A pressure-resistant container with an internal volume of 15 ml was loaded with 9.0 g of a 30% aqueous solution polystyrene sulfonic acid (Polysciences, Inc.; average molecular weight: 70,000) as a homogeneous acid catalyst and 1.0 g of pulverized palm EFB (obtained from Indonesia, dried, then pulverized with a cutter mill) as the raw materials polysaccharides, and hydrolysis reaction was performed at 90° C. for 2 hours. After the reaction, reaction solution and undegraded residues (lignin is the main constituent) were separated by filtration. When the reaction solution was analyzed by HPLC, the monosaccharides glucose, xylose and mannose were generated, the total yield thereof was 30% (which means 0.30 g of monosaccharides were obtained from 1.0 g of raw materials).

In addition, undegraded residues were washed with 5 ml of water and the wash was recovered. The recovered reaction solution and wash were placed into a centrifugal concentrator equipped with a separation membrane (Sartorius K.K., Vivaspin 20; inner volume: 20 ml; molecular weight cut-off: 10,000; membrane material: polyether sulfone; membrane surface area: 6.0 cm²) and subjected to a centrifuge (4,000 G, 10 minutes). At a stage where the solution was concentrated to approximately 5 ml, 10 ml of water was added and centrifugal separation treatment was carried out again. The same operation was further repeated twice, finally concentration was carried out to approximately 8 ml, approximately 35 ml of permeate containing mainly monosaccharides and 8 ml of concentrate containing mainly catalyst (approximately 8 g) were obtained. The catalyst concentration of the concentrate was 32%, which was a concentration of 1.1 times with respect to the initial solution (30%). The catalyst recovery ratio was 95%. The catalyst could be recovered at a high concentration and with a high recovery ratio.

8 g of concentrate containing the recovered catalyst was mixed as-is with 1.0 g of palm EFB to carry out a hydrolysis reaction again. The total yield of monosaccharides with a reaction at 90° C. for 2 hours was 30%, and it was found that the catalyst recovered by membrane separation could be recycled as-is without requiring a concentration operation or the like.

### Example 2

In a similar manner to Example 1, 9.0 g of 10% aqueous solution of lignin sulfonic acid (Aldrich; average molecular weight: 7,000; acid form converted from sodium salt form with an ion-exchange resin) as a homogeneous acid catalyst and 1.0 g of pulverized palm EFB were loaded, and hydrolysis reaction was performed at 120° C. for 2 hours. After the reaction, the reaction solution was separated by filtration from undegraded residues. The total yield of monosaccharides was 32%.

In addition, undegraded residues were washed with 5 ml of water and the wash was recovered. The recovered reaction solution and wash were placed into a centrifugal concentrator equipped with a separation membrane (molecular weight cut-off: 3,000) and subjected to a centrifuge (4,000 G, 10 minutes). In a similar manner to Example 1, monosaccharides and catalyst were separated by membrane separation. The liquid amount of the final catalyst concentrate was 5 ml (approximately 5 g), the catalyst concentration was 15%, which was a concentration of 1.5 times with respect to the initial solution (10%). The catalyst recovery ratio was 90%. The catalyst could be recovered at a high concentration and with a high recovery ratio.

### Example 3

A pressure-resistant glass bottle with an inner capacity of 50 ml was loaded with 20.0 g of 10% aqueous solution (pH 0.9) of phosphotungstic acid (manufactured by Nippon Inorganic Colour & Chemical Co., Ltd.; moisture content of approximately 16% as crystallization water; molecular weight without moisture: 2881) as a homogeneous acid catalyst and 4.0 g of microcrystalline cellulose Avicel (manufactured by Merck Ltd.), and saccharification reaction was performed at 150° C. for 6 hours while shaking with an oil shaker. The glucose yield was 37% and the glucose selectivity was 80%. After the reaction, the solids remaining without being dissolved were removed by centrifugal separation to obtain a reaction solution. The solids were further washed with approximately 50 g of water and a sample (saccharification solution A) was obtained by combining the wash with the reaction solution.

Subsequently, a separation step of monosaccharide and catalyst was performed. That is to say, 40.7 g of the above saccharification solution A (containing 1.4 g of phosphotungstic acid and 0.7 g of glucose) was added to a stirring separation membrane evaluation apparatus UHP-43K (manufactured by Advantec Co., Ltd.) equipped with the flat sheet membrane NTR-7450 (manufactured by Nitto Denko Corp.; material is sulfonated polyether sulfone, an organic polymer), a nano-filtration membrane serving as a molecular sieve membrane. Subsequently, the concentrate side (side containing saccharification solution A) was pressurized to 0.3 MPa to carry out membrane separation, and approximately 20 g of permeate was obtained on the permeation side across from the membrane. The operation of adding approximately 20 g of water to the concentrate, carrying out membrane separation and obtaining approximately 20 g of permeate was repeated twice, and ultimately 13.7 g of concentrate (catalyst recovery solution A) and a total of 63.8 g of permeate were obtained. The acid concentration of the concentrate was 10.2% and the acid concentration of the permeate was 0.004%. The catalyst recovery ratio was calculated to be 99.8% (based on permeate), which was found to be an extremely high recovery ratio. 91% of glucose was present in the permeate, it was found that the catalyst and glucose could be separated with a membrane.

Subsequently, a catalyst recycling step was carried out. A glass bottle was loaded with 10.0 g of the catalyst recovery solution A obtained earlier and 2.0 g of Avicel, and a hydrolysis reaction was performed at 150° C. for 6 hours. The glucose yield was 38% and selectivity was 78%, which were equivalent to the first reaction. From this, it was found that the catalyst recovered by membrane separation could be recycled as-is without undergoing a concentration operation or the like.

### Example 4

Similarly to Example 3, saccharification reaction with phosphotungstic acid as the catalyst and membrane separation experiment were carried out. However, this time, the flat sheet membrane NTR-7410 (manufactured by Nitto Denko Corp.), a nano-filtration membrane, was used as the molecular sieve membrane. As a result of carrying out the membrane separation step, 81% of catalyst was recovered in the concentrate and 92% of glucose was recovered in the permeate.

### Example 5

A cellulose saccharification reaction was carried out similarly to Example 3 but using 1% aqueous solution (pH 0.8) of polyvinyl sulfonic acid (manufactured by Aldrich; used after converting into acid form with an ion-exchange resin; average molecular weight: 2,000) as the catalyst. With a reaction at 165° C. for 1 hour, the glucose yield was 25% and selectivity was 80%. Furthermore similarly to Example 3, a membrane separation step was performed using NTR-7450. As a result, 73% of catalyst was recovered in the concentrate and 90% of glucose was recovered in the permeate.

### Example 6

A cellulose saccharification reaction was carried out similarly to Example 3 but using a 2% aqueous solution of poly(styrenesulfonic acid/maleic acid) (copolymer at 1:1 molar ratio; manufactured by Aldrich; used after converting into acid form with an ion-exchange resin; average molecular weight; 20,000) as the catalyst. With a reaction at 150° C. for 2 hours, the glucose yield was 22% and selectivity was 79%. Furthermore, similarly to Example 3, a membrane separation step was performed but this time the ultrafiltration capsule Minimate 65D (manufactured by Pall Corp.) equipped with an ultrafiltration membrane (Omega series 65D manufactured by Pall Corp.) was used during membrane separation. As a result of carrying out the membrane separation step, 84% of catalyst was recovered in the concentrate and 91% of glucose was recovered in the permeate.

### Example 7

Palm EFB demineralization and hemicellulose removal step was carried out. That is to say, 2.0 g of pulverized palm EFB (dry) and 20.0 g of an aqueous solution of 2% sulfuric acid were loaded into a pressure-resistant container and heated at 125° C. for 3 hours. Thereafter, the liquid content and the solid content were separated by filtration, furthermore, the solid content was washed with water. When the recovered filtrate was analyzed, the generation of 0.4 g of xylose and 0.03 g of glucose was observed. Meanwhile, the solid content (wet) was placed into a pressure-resistant container, 2.0 g of phosphotungstic acid was added as a catalyst, furthermore, water was added so as to reach 20.0 g in total reactants. This was heated at 150° C. for 6 hours while shaking with an oil shaker to perform a hydrolysis step. When the reaction solution was analyzed, the generation of 0.5 g of glucose was observed. Thereafter, the solid content was removed by similar methods to Example 3 to obtain a saccharification solution.

Subsequently, separation step of the monosaccharide and catalyst present in the saccharification solution was performed. The membrane separation was carried out in a similar manner to the method described in Example 3. That is to say, NTR-7450 (manufactured by Nitto Denko Corp.) was used as a molecular sieve membrane. As a result of membrane separation, 99.8% of phosphotungstic acid was recovered in the concentrate (catalyst recovery solution B) and 90% of glucose was recovered in the permeate. An extremely high catalyst recovery ratio was obtained.

### Example 8

Subsequently to Example 7, a catalyst recycling step was carried out. Palm EFB was prepared, which was treated for demineralization with entirely equivalent methods and amounts to Example 7, and mixed with the catalyst recovery solution B (phosphotungstic acid concentration: 10.4%) obtained earlier. When a hydrolysis reaction was performed at 150° C. for 6 hours, generation of 0.5 g of glucose was observed. From this, it was found that the catalyst recovered by membrane separation could be recycled as-is without undergoing a concentration operation or the like.

### Example 9

In a pressure-resistant container, 20.0 g of 10% aqueous solution of phosphotungstic acid and 2.0 g of Avicel were mixed, and a saccharification reaction was carried out at 150° C. The reaction solution was sampled over time to measure the glucose yield and selectivity. The results are shown along with the reaction conditions in Table 1. Subsequently, solid content was removed by filtration from the reaction solution after the reaction to obtain a saccharification solution. In addition, when a separation experiment of the catalyst and monosaccharide was carried out using the saccharification solution by a similar method to Example 3 by using NTR-7450 (manufactured by Nitto Denko Corp.) as the separation membrane, satisfactory separation results equivalent to Example 3 were obtained.

### Examples 10 to 14

Hydrolysis reaction of Avicel with phosphotungstic acid as the catalyst was carried out under various conditions. That is to say, it was performed with methods similar to Example 9, but the phosphotungstic acid concentration, the reaction temperature and the reaction time were changed to each of the conditions indicated in Table 1. The results are also shown along in Table 1. Although the glucose yield increases with reaction time selectivity decreases. This is due to excessive degradation occurring. From the results of Table 1, selectivity was found to be better when the catalyst concentration higher (Examples 9 and 10; comparison in the same extent of glucose yield). In addition, when the reaction temperature was higher, selectivity was also found to be better (comparison of Examples 10 and 11). Next, using the various saccharification solution obtained, when a membrane separation experiment for the catalyst and monosaccharide was carried out by similar methods to Example 3, satisfactory separation results equivalent to Example 3 were obtained.

### Comparative Example 1

Hydrolysis reaction of Avicel was carried out similarly to Example 9 but using 1% sulfuric acid as the catalyst. The reaction results are shown in Table 1. Compared to phosphotungstic acid, both selectivity and reaction speed were found to be low (comparison to Example 9 with the same extent of proton amount). Next, using the obtained saccharification solution, membrane separation experiment for the catalyst and monosaccharide was carried out by similar methods to Example 3. Sulfuric acid, which is the catalyst, and glucose were not separated at all, both permeated the membrane and were recovered on the permeation side.

TABLE 1

| Example/ | | | | | | |
|---|---|---|---|---|---|---|
| Comparative | Catalyst | Reaction | | | | |
| Example | concentration | Temperature | Reaction time | | Glucose yield | Selectivity |
| Example 9 | 10% | 150° C. | 1 | hour | 17% | 96% |
| | | | 3 | hours | 28% | 88% |
| | | | 6 | hours | 38% | 78% |
| Example 10 | 30% | 150° C. | 30 | minutes | 29% | 93% |
| | | | 1 | hour | 38% | 87% |
| | | | 2 | hours | 44% | 74% |
| Example 11 | 30% | 135° C. | 1 | hour | 17% | 97% |
| | | | 3 | hours | 30% | 89% |
| | | | 9 | hours | 40% | 71% |
| Example 12 | 10% | 180° C. | 10 | minutes | 50% | 75% |
| Example 13 | 5% | 185° C. | 10 | minutes | 47% | 78% |
| Example 14 | 1% | 200° C. | 5 | minutes | 21% | 93% |
| Comparative | 1% | 150° C. | 3 | hours | 19% | 86% |
| Example 1 | | | 8 | hours | 27% | 70% |

### Example 15

Saccharification reaction of palm EFB and catalyst recovery were carried out by the series of processes indicated below.

Pretreatment step (1) (hot water treatment): first, the operation of eliminating soluble salts by hot water treatment was carried out (desalting step). That is to say, 12.5 g of pulverized palm EFB (10% water content) and 50 g of ion exchanged water were loaded into a 100 ml pressure-resistant container, the container was sealed and heated at 150° C. for 30 minutes. Thereafter, the reaction solution and the solid residues (designated by residue A) were separated by filtration, furthermore, residue A was washed with 20 g of water twice. When the recovered reaction filtrate and the washes were analyzed by ICP, although no generation of monosaccharides was observed, elution of soluble salts such as potassium, sodium, calcium and magnesium was observed.

Pretreatment step (2) (dilute sulfuric acid treatment): subsequently, decomposition of hemicellulose by dilute sulfuric acid treatment was carried out (hemicellulose removal step). To the entire amount of residue A (25.6 g of water wet body), 0.25 g of sulfuric acid and 36.6 g of pure water were mixed (final concentration of sulfuric acid: 0.4%), and heated in a pressure-resistant container at 150° C. for 1 hour. Thereafter, the reaction solution and the solid residues (designated by residue B) were separated by filtration, furthermore, solid residue B was washed with 20 g of water twice. When the recovered reaction filtrate and the washes were analyzed, generation of 1.9 g of xylose, 0.1 g of glucose and 0.1 g of mannose was observed.

Saccharification step (heteropolyacid treatment): subsequently, cellulose saccharification reaction with heteropolyacid as catalyst was carried out. To the entire amount of residue B (21.6 g water wet), 3.75 g of phosphotungstic acid as the catalyst and 37.2 g of pure water were added (final concentration of catalyst: 6%), and heated at 175° C. for 3 hours. Thereafter, the reaction solution and the solid residues (designated by residue C) were separated by filtration, furthermore, residue C was washed with 20 g of water twice. When the recovered reaction filtrate and the washes (80.5 g in total) were analyzed, generation of a total of 1.8 g of glucose was observed. In addition, from the results of ICP measurements, 2.1 g in total of the catalyst phosphotungstic acid was found to be present in the reaction filtrate and the washes (55% amount of loaded catalyst). Meanwhile, when residue C was dried and phosphotungstic acid was quantified by ash amount measurement and fluorescence X-ray, 1.8 g of phosphotungstic acid was found to be present in residue C (45% amount of loaded catalyst). Phosphotungsten was found to be adsorbed on solid residues.

### Example 16

Catalyst recovery from reaction solution: using the reaction solution obtained in Example 15, the operation of recovering phosphotungstic acid from the reaction solution was carried out. That is to say, from the mixed solution of reaction filtrate and washes obtained in the heteropolyacid treatment of Example 15, 38 g (containing 1.0 g of phosphotungstic acid and 0.9 g of glucose) was subjected to membrane separation similarly to Example 3 using the separation membrane NTR-7450. However, the operation condition was room temperature and the operation pressure was 0.6 MPa. As a result, for phosphotungstic acid, 99% or greater was recovered on the concentration side and for glucose, 90% or greater was recovered on the permeation side. Ultimately phosphotungstic acid was concentrated to 8%.

### Example 17

Catalyst recovery from solid residues (organic compound thermal decomposition): catalyst recovery operation from residues by thermal decomposition of organic compound was carried out. That is to say, residue C obtained in Example 15 was dried (6.4 g dry weight), of which 0.5 g (containing 0.14 g of phosphotungstic acid) was taken to a calcination dish and heated in a muffle furnace at 450° C. for 1 hour. Note that air was supplied during heating. After heating, 0.15 g of brown residue was obtained. This residue was added with 1.0 g of pure water and stirred at room temperature for 30 minutes to elute water-soluble constituents and subjected to centrifugation, and the supernatant after centrifugation was recovered. This operation was further repeated twice and a total of approximately 3 g of eluate was obtained. When this eluate was analyzed by LC, 0.11 g of phosphotungstic acid was observed (recovery ratio: 85%). The retention time in the LC analysis was the same as a fresh catalyst, and no structural alteration was observed. From this, it was found that the catalyst phosphotungstic acid could be recovered from solid residues by thermally decomposing the organic compound.

### Examples 18 to 22

Catalyst recovery from residue C was attempted under various temperature conditions. The recovery experiment was carried out entirely similarly to Example 17, but the heating temperature and time were changed to the conditions indicated in Table 2. The recovery ratio of phosphotungstic acid is shown along in Table 2.

Note that in Examples 21 and 22, which uses high temperature conditions, there was almost no recovery as phosphotungsten. It was found that under high temperature conditions, phosphotungstic acid undergoes dehydration, generating tungsten trioxide. Thus, when the residues after heating were treated with alkali (aqueous solution of 1% sodium hydroxide), it was found that it was eluted as tungstate ion and that it could be recovered.

TABLE 2

| | Heating | | Phosphotungstic | |
|---|---|---|---|---|
| | tem- | Heating | acid | |
| Example | perature | time | recovery ratio | Remarks |
| Example 17 | 450° C. | 1 hour | 85% | — |
| Example 18 | 450° C. | 3 hours | 90% | — |
| Example 19 | 400° C. | 1 hour | 20% | — |
| Example 20 | 500° C. | 1 hour | 70% | — |
| Example 21 | 600° C. | 1 hour | 1% | Phosphotungstic |
| | | | | acid recovered |
| | | | | with alkali |
| | | | | treatment |
| Example 22 | 1000° C. | 1 hour | 0% | Phosphotungstic |
| | | | | acid recovered |
| | | | | with alkali |
| | | | | treatment |

### Example 23

Catalyst recovery from solid residues (organic solvent elution): catalyst elution experiment from residues by organic solvent treatment was carried out. That is to say, 0.1 g of dry body of residue C (containing 0.027 g of phosphotungstic acid) obtained in Example 15 was mixed with 1 ml of an aqueous solution of 50% acetone and stirred at room temperature for 30 minutes. Thereafter, solid-liquid separation was carried out by centrifugal separation and a supernatant (eluate) and solid residues were obtained. Similar operation was repeated twice to carry out elution and a total of approximately 3 ml of eluate was obtained. When the eluate was analyzed by LC, 0.023 g of phosphotungstic acid was observed (recovery ratio: 85%). From this, it was found that the catalyst phosphotungstic acid could be recovered by acetone elution.

### Examples 24 to 28

Catalyst elution experiments were carried out with various eluents to investigate the influence of solvent species. Experiments were carried out entirely similarly to Example 23 except that various eluents were used instead of an aqueous solution of 50% acetone. In order to clearly establish the solvent species differences, phosphotungstic acid elution ratios were compared at the time point of the end of one elution operation. The results are shown in Table 3. Note that, in the alkali treatment of Example 28, elution was found to be as tungstate ion.

### Comparative Examples 2 and 3

Catalyst elution experiments were carried out similarly to Example 23, using water and an aqueous solution of 1% sulfuric acid. The results are shown together in Table 3. With water and sulfuric acid, the catalyst was found to almost not elute.

TABLE 3

| | | Phosphotungstic |
|---|---|---|
| | | acid |
| Example/ | | elution ratio |
| Comparative | | (one elution |
| Example | Eluent | operation) |
| Example 23 | Aqueous solution of 50% acetone | 42% |
| Example 24 | Aqueous solution of 50% | 50% |
| | tetrahydrofuran | |
| Example 25 | Aqueous solution of 50% diethylene | 45% |
| | glycol dimethyl ether | |
| Example 26 | Aqueous solution of 50% ethanol | 15% |
| Example 27 | Aqueous solution of 80% acetone | 45% |
| Example 28 | Aqueous solution of 1% sodium | 81% |
| | hydroxide | |
| Comparative | Water | 3% |
| Example 2 | | |
| Comparative | Aqueous solution of 1% sulfuric acid | 0% |
| Example 3 | | |

### Example 29

Saccharification experiments of palm EFB were carried out by the series of processes indicated below.

Pretreatment step (1): dilute sulfuric acid treatment for the purpose of elimination of soluble salts and hemicellulose degradation were carried out (hemicellulose removal step). That is to say, 24.0 g of pulverized palm EFB (10% aqueous body) and 120 g of an aqueous solution of 1% sulfuric acid were loaded into a 200 ml pressure-resistant container, the container was sealed and heated at 150° C. for 1 hour. When the reaction solution was analyzed by LC, generation of 4.8 g of xylose, 0.2 g of glucose and 0.2 g of mannose (total monosaccharide yield was 24%) was observed. Thereafter, the reaction solution and solid residues were separated by filtration, furthermore, the residues were washed with 200 g of water three times. When the residues after washing were subjected to vacuum drying (70° C., 2 hours), 15.4 g of solid (pretreated EFB-1) was obtained (weight yield based on dry body: 71%).

Pretreatment step (2): subsequently, acetone treatment for the purpose of elimination of lignin was carried out (delignification step). That is to say, from the obtained pretreated EFB-1, 1.0 g was fractionated, mixed with 10 ml of an aqueous solution of 50% acetone, loaded into a 50 ml pressure-resistant container, and heat treatment at 120° C. for 2 hours was performed. Thereafter, solid-liquid separation was carried out by filtration, and solid residues were washed with 30 ml of pure water three times. Then, after being subjected to vacuum drying, 0.81 g of solid (pretreated EFB-2) was obtained.

Catalyst adsorption experiment: the entire amount of pretreated EFB-2 obtained in the above step was mixed with 10 ml of an aqueous solution of 1% phosphotungstic acid and heated at 150° C. for 30 minutes. After letting stand still, a small amount of supernatant was subjected to LC analysis, the concentration of free phosphotungstic acid was determined, and amount of phosphotungstic acid adsorbed to pretreated EFB was calculated. As a result, the adsorption ratio was 58%.

Cellulose saccharification experiment: after the above catalyst adsorption experiment, 0.4 g of phosphotungstic acid was added to the reaction solution, so as to bring the catalyst concentration to 5%. Then, heating at 150° C. for 12 hours, saccharification reaction of cellulose was carried out. The amount of glucose generated was 0.16 g.

Catalyst recovery experiment: catalyst recovery from the reaction solution was carried out. That is to say, the saccharification reaction solution obtained in the above saccharification experiment was subjected to solid-liquid separation by filtration, and solid residues were washed with 20 ml of pure water twice. The reaction filtrate and the washes were mixed, from which 40 g was used to carryout phosphotungstic acid recovery experiment similarly to Example 3 with the separation membrane NTR-7450. The recovery ratio of the catalyst was 99% or greater.

### Examples 30 to 34

A series of experiments were carried out similarly to Example 29 but changing the conditions of pretreatment step (2). Instead of 50% acetone, a variety of treatment solutions and treatment conditions indicated in Table 4 were used. However, in Example 34, catalyst adsorption experiment was carried out immediately using 1.0 g of pretreated EFB-1 without carrying out pretreatment step (2). These results are shown in Table 4. It was found that performing treatments that eliminate lignin, such as organic solvent treatment and alkali treatment, decreases the catalyst adsorption ratio.

TABLE 4

| | | | Catalyst adsorption | Saccharification experiment | Catalyst recovery |
|---|---|---|---|---|---|
| | Treatment solution in | Treatment conditions in | experiment result, | result, Glucose generation | experiment result, |
| Example | pretreatment step (2) | pretreatment step (2) | Catalyst absorption ratio | amount | Catalyst recovery ratio |
| Example 29 | Aqueous solution of 50% | 120° C., 2 hours | 58% | 0.16 g | >99% |
| | acetone | | | | |
| Example 30 | Aqueous solution of 50% | 120° C., 2 hours | 35% | 0.18 g | >99% |
| | acetone containing 1% | | | | |
| | sulfuric acid | | | | |
| Example 31 | Aqueous solution of 50% | 120° C., 2 hours | 37% | 0.13 g | >99% |
| | acetone containing 1% | | | | |
| | sodium hydroxide | | | | |
| Example 32 | Aqueous solution of 1% | 150° C., 2 hours | 16% | 0.11 g | >99% |
| | sodium hydroxide | | | | |
| Example 33 | Aqueous solution of 2.5% | 150° C., 3 hours | 38% | Not performed | Not performed |
| | sulfuric acid | | | | |
| Example 34 | Not treated | — | 63% | 0.16 g | >99% |

### Comparative Example 4

A saccharification experiment was carried out similarly to Example 29 but using sulfuric acid as the catalyst. That is to say, after performing up to pretreatment step (2) similarly to Example 29, 10 ml of an aqueous solution of 5% sulfuric acid was added to pretreated EFB-2 and saccharification reaction was carried out by heating at 150° C. for 12 hours. The amount of glucose generated was 0.08 g. Subsequently, a catalyst recovery experiment using NTR-7450 was carried out; however, the catalyst sulfuric acid was not recovered at all.

### Examples 35 to 37

Experiments were carried out similarly to Example 34 using a variety of heteropolyacids as the catalyst. That is to say, experiments were carried out similarly to Example 34 (without carrying out pretreatment step (2)) but using the heteropolyacids indicated in Table 5 instead of phosphotungstic acid as the catalyst in the catalyst adsorption experiment and cellulose saccharification experiment. The results are shown together in Table 5. Note that silicotungstic acid and phosphomolybdic acid were manufactured by Nippon Inorganic Colour & Chemical Co., Ltd., and borotungstic acid was a prepared product.

### Example 38

An experiment was carried out similarly to Example 34 using polyvinyl sulfonic acid. That is to say, 1.0 g of pretreated EFB-1 obtained in Example 29 was fractionated, 10 ml of 2.5% polyvinyl sulfonic acid (used in Example 5) was added, and saccharification reaction was carried out at 150° C. for 6 hours. Subsequently, after solid-liquid separation, recovery experiment of the catalyst constituent present in the liquid was carried out. The results are shown together in Table 5. Note that no catalyst adsorption experiment was carried out.

### Example 39

An experiment was carried out similarly to Example 38 using a copolymer of vinyl sulfonic acid and acrylic acid. The saccharification reaction was entirely similar to Example 38 but a copolymer of vinyl sulfonic acid and acrylic acid was used as the catalyst instead of polyvinyl sulfonic acid. The results are shown together in Table 5.

Note that preparation of the copolymer was carried out as follows. That is to say, 60 g of an aqueous solution of 25% sodium vinyl sulfonate and 7.3 g of an aqueous solution of 37% sodium acrylate were mixed in a flask (molar ratio was 8 versus 2), furthermore, 106.9 g of pure water was added and heated to 80° C. Next, 2.9 g of an aqueous solution of 10% sodium persulfate was added to the mixture and the mixture was maintained at an internal temperature of 80° C. to 90° C. for 1 hour to let the polymerization reaction proceed. As a result of GPC analysis, a polymer with an average molecular weight of approximately 3,000 was generated. This polymer was converted into acid form with an ion-exchange resin, and used as the catalyst.

TABLE 5

| | | Adsorption | Saccharification | Catalyst recovery |
|---|---|---|---|---|
| | | experiment result, | experiment result, | experiment result, |
| | | Catalyst adsorption | Glucose generation | Catalyst recovery |
| Example | Catalyst | ratio | amount | ratio |
| Example 34 | Phosphotungstic acid | 63% | 0.16 g | >99% |
| Example 35 | Silicotungstic acid | 32% | 0.15 g | >99% |
| Example 36 | Borotungstic acid | 45% | 0.16 g | >99% |
| Example 37 | Phosphomolybdic acid | 30% | 0.10 g | >90% |
| Example 38 | Polyvinylsulfonic acid | Not performed | 0.14 g | 85% |
| Example 39 | Poly(vinylsulfonic | Not performed | 0.18 g | 86% |
| | acid/acrylic acid) | | | |

### Example 40

Saccharification reaction of palm EFB and catalyst recovery were carried out by the series of processes indicated below.

Pretreatment step (hot water treatment): with 12.5 g of ground palm EFB (10% aqueous body) as raw materials, hot water treatment was carried out entirely similarly to Example 15 (desalting step).

Saccharification step (1): subsequently, degradation of hemicellulose by phosphotungstic acid was carried out. To the residues after hot water treatment (24.9 g of water wet), 35 g of pure water and 2.5 g of phosphotungstic acid were added, and the mixture was heated in a pressure-resistant container at 150° C. for 1 hour. Thereafter, the reaction solution and solid residues were separated by filtration, furthermore, the solid residues were washed with 30 g of water twice. When the recovered reaction filtrate and washes were analyzed, generation of 2.7 g of xylose, 0.1 g of glucose and 0.1 g of mannose was observed.

Saccharification step (2): subsequently, degradation of cellulose by phosphotungstic acid was carried out. To the entire amount of the solid residues obtained in saccharification step (1) (20.8 g of water wet body), 25 g of pure water and 2.5 g of phosphotungstic acid were added, and the mixture was heated at 180° C. for 3 hours. Thereafter, the reaction solution and solid residues were separated by filtration, furthermore, the solid residues were washed with 30 g of pure water twice. When the recovered reaction filtrate and washes were analyzed, generation of 2.3 g of glucose was observed.

Catalyst recovery from the reaction solution: reaction solutions and washes obtained in saccharification steps (1) and (2) were all mixed, of which 40 g (containing 1.1 g of phosphotungstic acid, 0.5 g of xylose and 0.5 g of glucose) was fractionated, and membrane separation was carried out similarly to Example 3 using the separation membrane NTR-7450. However, the operation condition was room temperature and the operation pressure was 0.6M Pa. As a result, for phosphotungstic acid, 99.8% was recovered on the concentrate side and for xylose and glucose, 91% was recovered on the permeate side.

In the following examples and comparative examples measurements were carried out as follows:

(1) Membrane Permeation Rate of the Permeate:

Membrane permeation rate was determined by measuring the flow rate of the permeate during membrane separation.

(2) Quantification of Phosphotungstic Acid:

The tungsten amount was determined by inductively coupled plasma analysis (ICP analysis) using the apparatus mentioned below and the phosphotungstic acid amount was calculated.

Apparatus: ICPE-9000 (product name, manufactured by Shimadzu Corp.)

(3) Quantification of Glucose:

The quantification of glucose was carried out using liquid chromatography (HPLC) LC-8020 (manufactured by Tosoh Corp.) under the following conditions:

Measurement Conditions:

Column: TSK-GEL Amide80 (product name, manufactured by Tosoh Corp.)

Column temperature: 60° C.

Mobile phase: acetonitrile-water mixed solvent (volume ratio: 75/25)

Detector: RI

In the following examples and comparative examples, evaluation was carried out with the calculation formulae below:

(1) Initial Phosphotungstic Acid Rejection Ratio:

The initial phosphotungstic acid rejection ratio represents the phosphotungstic acid rejection ratio when the permeate amount has reached 10% of the liquid amount of the solution subjected to membrane separation.

Phosphotungstic acid rejection ratio (%)=[{(phosphotungstic acid concentration of the solution subjected to membrane separation)−(phosphotungstic acid concentration of the permeate)}/(phosphotungstic acid concentration of the solution subjected to membrane separation)]×100

(2) Glucose Permeation Ratio:

Glucose permeation ratio (%)={(glucose concentration of permeate)/(glucose concentration of the solution subjected to membrane separation)}×100

Heteropolyacid Adsorption Experiment with Metal Oxide

### Comparative Example 5

When phosphotungstic acid concentration was measured in a solution after 1 g of γ-alumina manufactured by Saint-Gobain NorPro (product name “SA6576”) was added to 30 g of an aqueous solution of 15.5% phosphotungstic acid and immersed for 1 hour, the phosphotungstic acid concentration dropped to 14.2%. It was verified that 8.4% of the loaded phosphotungstic acid was adsorbed to γ-alumina.

Note that phosphotungstic acid (product name, manufactured by Nippon Inorganic Colour & Chemical Co., Ltd.) was used as the phosphotungstic acid.

Heteropolyacid Adsorption Experiment with Metal Oxide

### Comparative Examples 6 to 9

Adsorption experiments were carried out similarly to Comparative Example 5 using the metal oxides indicated in Table 6 as the metal oxide.

Heteropolyacid Adsorption Experiment with Organic Polymer Membrane

### Examples 41 to 44

Adsorption experiments were carried out similarly to Comparative Example 5 using the organic polymer membranes indicated in Table 6 instead of the metal oxide.

The results of the heteropolyacid adsorption experiments are shown in Table 6.

TABLE 6

| | | | | Phosphotungstic acid | | |
|---|---|---|---|---|---|---|
| | | | | concentration | | |
| | Adsorption material | | | | After | Loss due to |
| | Species | Manufacturer | Product No. | Initial (%) | adsorption (%) | adsorption (%) |
| Comparative | γ-alumina | NORPRO | SA6576 | 15.5 | 14.2 | 8.4 |
| Example 5 | | | | | | |
| Comparative | α-alumina | NORPRO | SA6576(baked at | 15.5 | 14.3 | 7.7 |
| Example 6 | | | 1000° C.) | | | |
| Comparative | Titania | NORPRO | ST61120 | 15.5 | 14.8 | 4.5 |
| Example 7 | | | | | | |
| Comparative | Zirconia | NORPRO | XZ16154 | 15.5 | 14.7 | 5.2 |
| Example 8 | | | | | | |
| Comparative | Silica | Fuji Silysia | Q-30 | 15.5 | 15.1 | 2.6 |
| Example 9 | | | | | | |
| Example 41 | Organic polymer nano- | Nitto Denko | NTR-7450 | 15.5 | 15.5 | 0 |
| | filtration membrane | | | | | |
| Example 42 | Organic polymer nano- | KOCH | SelRO MPS-34 | 15.5 | 15.5 | 0 |
| | filtration membrane | | | | | |
| Example 43 | Organic polymer nano- | GE | Desal DL | 15.5 | 15.5 | 0 |
| | filtration membrane | | | | | |
| Example 44 | Organic polymer nano- | GE | GH | 15.5 | 15.5 | 0 |
| | filtration membrane | | | | | |

Heteropolyacid Separation Experiment

### Example 45

A heteropolyacid separation experiment was carried out using the separation membrane evaluation apparatus Membrane Master C10-T (manufactured by Nitto Denko Corp.; membrane surface area: 60 cm²) equipped with a flat membrane NTR-7450 (manufactured by Nitto Denko Corp.) that is a nano-filtration membrane. By supplying a liquid for separation to this Membrane Master C10-T with a solution-sending pump, a flow of liquid parallel to the membrane is created, and separation membrane evaluation in cross-flow type becomes possible. Added was 100 g of liquid for separation (containing 4 g of phosphotungstic acid (manufactured by Nippon Inorganic Colour & Chemical Co., Ltd.; product name “phosphotungstic acid”) and 10 g of glucose (manufactured by Kanto Chemical Co., Inc.; product name “D(+)-glucose”)) subjected to membrane separation. Subsequently, concentrate side (side containing solution subjected to membrane separation) was pressurized to 0.3 MPa, and membrane separation was carried out at a temperature of 25° C., feeding flow rate of 100 ml/minute. The membrane permeation rate of the permeate while carrying out membrane separation was 105 g/min/m². Then, 50 g of permeate was obtained on the permeation side across from the membrane.

Initial phosphotungstic acid rejection ratio was 99.8% and the glucose permeation ratio was 99%.

Heteropolyacid Separation Experiment

### Examples 46 to 54

Separation experiments were carried out similarly to Example 45 except that the separation conditions were modified as in Table 7.

The results of the heteropolyacid separation experiments are shown in Table 7.

Note that the abbreviations in Table 6 and Table 7 are as follows:

### NORPRO: Saint-Gobain NorPro

### KOCH: Koch Membrane

### GE: GE Water & Process Technologies

NF: organic polymer nano-filtration membrane
UF: organic polymer ultrafiltration membrane

TABLE 7

| | Separation membrane | | | | | | | | Initial phos- | |
|---|---|---|---|---|---|---|---|---|---|---|
| | | | | Pure water | Load composition | | | Perme- | photungstic | |
| | | | | permeation rate | Phospho- | | Operation | ation | acid perme- | Glucose |
| | | | Spe- | (25° C., 0.1 | tungstic | Glucose | pressure | rate (g/ | ation blocking | permeation |
| | Manufacturer | Product No. | cies | MPa) (g/min/m²) | acid (%) | (%) | (MPa) | min/m²) | ratio (%) | ratio (%) |
| Example 45 | Nitto Denko | NTR-7450 | NF | 275 | 4 | 10 | 0.3 | 105 | 99.8 | 99 |
| Example 46 | Nitto Denko | NTR-7450 | NF | 275 | 9 | 10 | 0.3 | 70 | 97.5 | 100 |
| Example 47 | Nitto Denko | NTR-7450 | NF | 275 | 9 | 10 | 0.5 | 175 | 98.0 | 98 |
| Example 48 | Nitto Denko | NTR-7450 | NF | 275 | 9 | 10 | 0.6 | 222 | 98.1 | 100 |
| Example 49 | Nitto Denko | NTR-7450 | NF | 275 | 8 | 11 | 0.6 | 350 | 99.5 | 98 |
| Example 50 | KOCH | SelRO MPS-34 | NF | 21 | 2 | 10 | 0.6 | 68 | 99.9 | 100 |
| Example 51 | GE | Desal DL | NF | 17 | 2 | 10 | 0.6 | 67 | 99.9 | 98 |
| Example 52 | GE | GE | UF | 11 | 8 | 9 | 0.6 | 89 | 93.2 | 95 |
| Example 53 | GE | GH | UF | 23 | 10 | 13 | 0.6 | 74 | 99.2 | 100 |
| Example 54 | GE | GK | UF | 32 | 8 | 9 | 0.6 | 150 | 96.4 | 95 |

From the results of Examples 1 to 14 and Comparative Example 1, it was observed that generating monosaccharides through hydrolysis of polysaccharides using a homogeneous acid catalyst and performing membrane separation on the obtained reaction solution to separate the monosaccharides and the catalyst allows monosaccharides to be obtained with high yield and at the same time, the catalyst to be recovered with a high recovery ratio. In addition, it was observed that if the reaction time of the hydrolysis reaction becomes longer, overdegradation of monosaccharides occurs, and the yield for the monosaccharides becomes higher, but the selectivity for the monosaccharides decreases, and that the higher the catalyst concentration during the hydrolysis reaction and the higher the reaction temperature, the higher the selectivity becomes.

From the results of Examples 15 to 22, it was observed that generating monosaccharides through hydrolysis of polysaccharides using a homogeneous acid catalyst, subjecting the obtained reaction solution to solid-liquid separation to remove the reaction residues and thermally decomposing these also allow the catalyst to be recovered with a high recovery ratio.

From the results of Examples 23 to 28 and Comparative Examples 2 and 3, it was observed that generating monosaccharides through hydrolysis of polysaccharides using a homogeneous acid catalyst, subjecting the obtained reaction solution to solid-liquid separation to remove the reaction residues and adding an eluent to these residues allow the catalyst to be eluted and recovered with a high recovery ratio.

From the results of Examples 29 to 34 and Comparative Example 4, it was observed that carrying out dilute sulfuric acid treatment and acetone treatment as treatments to eliminate lignin from the polysaccharides prior to being subjected to hydrolysis, and then adding a homogeneous acid catalyst to carry out the hydrolysis allow the adsorption ratio of the catalyst to lignin to be restricted, and it was also observed that the conditions of the acetone treatment influence the adsorption ratio. In addition, also in Example 29 where the adsorption ratio was relatively high, it was observed that a high catalyst recovery ratio could be obtained by solid-liquid separation, washing of the reaction residues and membrane separation through a molecular sieve membrane of the reaction solution to which the wash was added. Furthermore, when a homogeneous acid catalyst with a molecular weight of 200 or greater was not used, no catalyst was recovered.

From the results of Examples 35 to 39, it was observed that when a variety of species of compounds are used as homogeneous acid catalysts and only dilute sulfuric acid treatment is carried out as pretreatment without carrying out acetone treatment, although differences in catalyst adsorption ratio are observed depending on the compound, the catalyst could be recovered with a high recovery ratio by membrane separation through a molecular sieve membrane, similarly to Example 29.

From the results of Example 40, it was observed that carrying out hot water treatment as a pretreatment of polysaccharides prior to being subjected to hydrolysis, adding a homogeneous acid catalyst to the polysaccharides after the pretreatment to carry out the hydrolysis reaction, and then, to the reaction residues obtained by carrying out solid-liquid separation, further adding a homogeneous acid catalyst to carry out a second hydrolysis allows more monosaccharides to be prepared. In addition, it was observed that a high catalyst recovery ratio could be obtained by gathering the solutions obtained in the two hydrolyses and carrying out solid-liquid separation, washing of the reaction residues and membrane separation through a molecular sieve membrane of the reaction solution to which the wash was added.

Note that although examples were shown in the above Examples 1 to 40, in which specific homogeneous acid catalysts and polysaccharides were used to carry out hydrolysis and catalyst separation, the mechanisms for separating the homogeneous acid catalyst from the homogeneous acid catalyst-containing solution after the hydrolysis reaction are all similar, it can be stated from the results of the above Examples 1 to 40 and Comparative Examples 1 to 4 that the monosaccharide preparation method of the present invention can be applied in a variety of modes described herein, allowing advantageous effects to be exerted.

In addition, from the results of Table 6, it was observed that, in contrast to the metal oxide constituting the inorganic membrane adsorbing phosphotungstic acid, the organic polymer membrane does not adsorb phosphotungstic acid. From this, it was found that the loss of phosphotungstic acid due to the metal oxide adsorbing phosphotungstic acid, which is a problem when separating phosphotungstic acid using an inorganic membrane, can be suppressed by using an organic polymer membrane.

From the results of Table 7, it was found that, by carrying out membrane separation using an organic polymer membrane with a pure water permeation rate of 1 g/min/m² or greater at 25° C. and 0.1 MPa for the separation of a heteropolyacid from the heteropolyacid-containing solution, even when the heteropolyacid concentration in the heteropolyacid-containing solution is high, permeation of the heteropolyacid can be rejected with an extremely high rejection ratio, allowing the heteropolyacid to be separated highly efficiently. Then, it was found that when glucose is contained in the heteropolyacid-containing solution, heteropolyacid and glucose can be separated sufficiently.

Note that, although examples were shown in the above Example 41 and onwards, in which specific organic polymer membranes were used, heteropolyacid was used as homogeneous acid catalyst to carry out membrane separation, the mechanisms whereby organic polymer membranes separate the homogeneous acid catalyst from the homogeneous acid catalyst-containing solution are all similar, it can be stated from the results of the above Examples 41 to 54 and Comparative Examples 5 to 9, the homogeneous catalyst separation method of the present invention can be applied in a variety of modes described herein, allowing advantageous effects to be exerted.

1. A method for preparing a monosaccharide by hydrolyzing a polysaccharide using a homogeneous acid catalyst, wherein the method comprises a hydrolysis step of hydrolyzing a polysaccharide using a homogeneous acid catalyst with a molecular weight of 200 or greater to generate a monosaccharide, and a separation step of the homogeneous acid catalyst after hydrolysis, and the separation step includes at least one step selected from the group consisting of the following (A) to (C):
(A) a step of separating the homogeneous acid catalyst by performing homogeneous acid catalyst membrane separation treatment using a molecular sieve membrane, on a homogeneous acid catalyst-containing solution after the hydrolysis step;
(B) a step of separating the homogeneous acid catalyst by performing organic compound thermal decomposition treatment on a hydrolysis reaction residue separated by solid-liquid separation after the hydrolysis step; and
(C) a step of separating the homogeneous acid catalyst by performing homogeneous acid catalyst elution treatment using an alkaline solution or an organic solvent-containing solution, on the hydrolysis reaction residue separated by solid-liquid separation after the hydrolysis step.

2. The method for preparing a monosaccharide according to claim 1, wherein the hydrolysis step is a step of carrying out hydrolysis with a proportion in mass between the homogeneous acid catalyst and water present in a reaction system in the range of 0.1:99.9 to 50:50, during the hydrolysis reaction.

3. The method for preparing a monosaccharide according to claim 1, wherein the homogeneous acid catalyst includes an organic compound having a sulfonic acid group and/or a heteropolyacid.

4. The method for preparing a monosaccharide according to claim 1, wherein the homogeneous acid catalyst includes a heteropolyacid.

5. The method for preparing a monosaccharide according to claim 1, wherein the method comprises a recycling step of recovering and recycling the homogeneous acid catalyst separated in the separation step.

6. The method for preparing a monosaccharide according to claim 5, wherein the recycling step is carried out immediately after the separation step in the monosaccharide preparation method.

7. The method for preparing a monosaccharide according to claim 1, wherein hydrolysis is carried out at a reaction temperature of 100° C. or higher in the hydrolysis step.

8. The method for preparing a monosaccharide according to claim 1, wherein the polysaccharide is a polysaccharide obtained through a pretreatment step including at least one among a desalting step, a delignification step and a hemicellulose removal step.

9. The method for preparing a monosaccharide according to claim 1, wherein the molecular sieve membrane used in the step of separating the homogeneous acid catalyst by performing the membrane separation treatment is a molecular sieve membrane using an organic polymer membrane and a pure water permeation rate of the organic polymer membrane is 1 g/min/m² or greater at 25° C. and 0.1 MPa.

10. The method for preparing a monosaccharide according to claim 1, wherein the organic polymer membrane is a nano-filtration membrane or an ultrafiltration membrane.

11. The method for preparing a monosaccharide according to claim 1, wherein the organic polymer membrane is a polymer membrane having a cation-exchange group.

12. The method for preparing a monosaccharide according to claim 11, wherein the organic polymer membrane is a polymer membrane having a sulfonic acid group.

13. A method for separating a homogeneous acid catalyst from a homogeneous acid catalyst-containing solution, wherein the method comprises a step of separating the homogeneous catalyst by performing homogeneous catalyst membrane separation treatment using a molecular sieve membrane, and the molecular sieve membrane is a molecular sieve membrane using an organic polymer membrane, and a pure water permeation rate of the organic polymer membrane is 1 g/min/m² or greater at 25° C. and 0.1 MPa.

14. The method for separating a homogeneous acid catalyst according to claim 13, wherein the organic polymer membrane is a nano-filtration membrane or an ultrafiltration membrane.

15. The method for separating a homogeneous acid catalyst according to claim 13, wherein the organic polymer membrane is a polymer membrane having a cation-exchange group.

16. The method for preparing a monosaccharide according to claim 2, wherein the homogeneous acid catalyst includes an organic compound having a sulfonic acid group and/or a heteropolyacid.

17. The method for preparing a monosaccharide according to claim 2, wherein the homogeneous acid catalyst includes a heteropolyacid.

18. The method for preparing a monosaccharide according to claim 3, wherein the homogeneous acid catalyst includes a heteropolyacid.

19. The method for preparing a monosaccharide according to claim 2, wherein the method comprises a recycling step of recovering and recycling the homogeneous acid catalyst separated in the separation step.

20. The method for preparing a monosaccharide according to claim 3, wherein the method comprises a recycling step of recovering and recycling the homogeneous acid catalyst separated in the separation step.
